(19) United States
(12) Patent Application Publication
(10) Pub. No.: US 2011/0028554 A1
(43) Pub. Date: Feb. 3, 2011

(54) PHARMACEUTICAL COMPOSITIONS COMPRISING DIIODOTHYRONINE AND THEIR THERAPEUTIC USE

(75) Inventors: Xavier Leverve, La Terrasse (FR); Nellie Taleux, Meylan (FR); Roland Favier, Jonage (FR); Michelle Favier, Jonage (FR); Franck Favier, Lyon (FR); Boris Favier, Lyon (FR); Yann Favier, Jonage (FR)

Correspondence Address: YOUNG & THOMPSON, 209 Madison Street, Suite 500, Alexandria, VA, 22314, US

(73) Assignees: UNIVERSITE JOSEPH FOURIER, GRENOBLE CEDEX 09 (FR); CENTRE NATIONAL DE LA RECHERCHE SCIENTIFIQUE, PARIS CEDEX 16 (FR); INSERM (INSTITUT NATIONAL DE LA SANTE ET DE LA RECHERCHE SCIENTIFIQUE), Paris Cedex (FR)

(21) Appl. No.: 12/600,109

(22) PCT Filed: May 16, 2008
(86) PCT No.: PCT/EP2008/056074

§ 371 Date: Oct. 21, 2010

(30) Foreign Application Priority Data
May 16, 2007 (FR) 07290634.0

(51) Int. Cl. A61K 31/198; A61P 3/00

(52) U.S. Cl. 514/567

(57) ABSTRACT

The present invention relates to a pharmaceutical composition comprising, as active substance, at least one hormone chosen among 3,5-diiodothyronine (3,5-T2), 3′,3-diiodothyronine (3′,3-T2), 3′,5-diiodothyronine (3′,5-T2), 3′-iodothyronine (3′-T), 3-iodothyronine (3-T) or 5-iodothyronine (5-T), in association with a pharmaceutically acceptable vehicle.

The present invention relates to new pharmaceutical compositions comprising diiodothyronine and their therapeutic use.

Thyroid hormones have been known for a long time. The thyroid hormone family consists in T4 hormone and the derived iodothyronines resulting from successive monodeiodinations of T4. The pathways of the deiodination cascade of T4 have been described by Hulbert A. J. (Biol. Rev., 2000). T4 gives T3 via an outer ring 5′-deiodination or rT3 via an inner ring 5′-deiodination. T3 results in 3,5-T2 via an outer ring 5′-deiodination or 3,3′-T2 via an inner ring 5′-deiodination, rT3 results in 3,3′-T2 via an outer ring 5′-deiodination or 3′,5′-T2 via an inner ring 5′-deiodination. 3-T1 is obtained via an inner ring 5′-deiodination from 3,5-T2 or via an outer ring 5′-deiodination from 3,3′-T2. 3′-T1 is obtained via an inner ring 5′-deiodination from 3,3′-T2 or via an outer ring 5′-deiodination from 3′,5′-T2.

For information, table 1 indicates the formula of several members of the thyroid hormone family.

TABLE 1
Formula of iodothyronine hormones

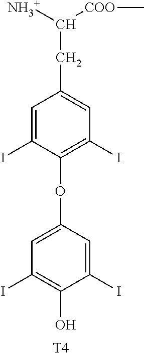

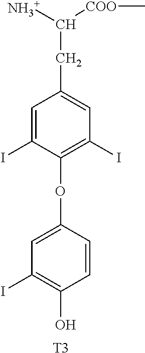

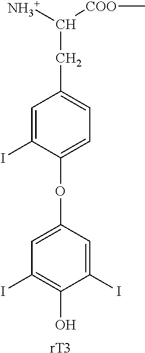

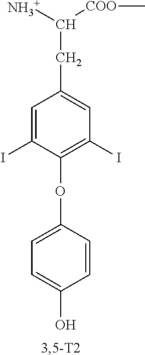

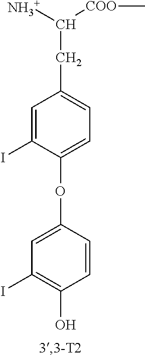

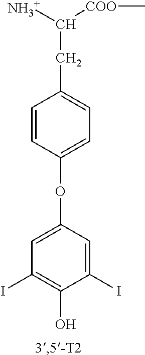

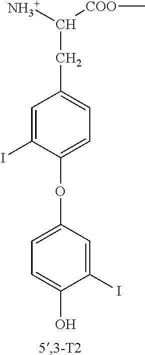

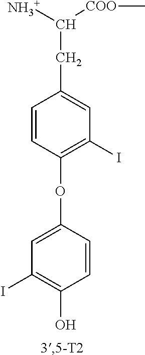

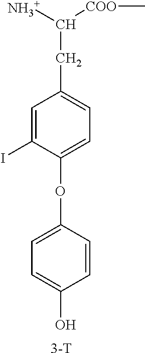

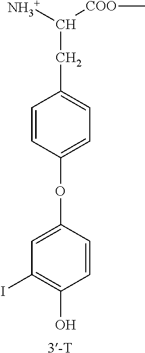

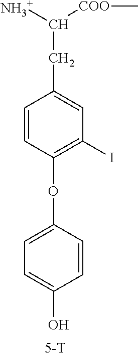

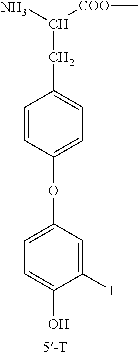

The known effects of thyroid hormones, particularly of the T3 hormone, result mainly from the binding to two nuclear receptors of the thyroid hormones, TRα-1 and TRβ-1 belonging to the family of nuclear receptors TRα and TRβ, which are supposed to have different effects. These receptors are thought to be highly specific towards T3, particularly relating to the number of iodine and the spatial arrangement (Bolger et al., J. Biol. Chem., 1980; Koerner et al., J. Biol. Chem., 1975; Dietrich et al., J. Med. Chem., 1977). Since the discovery of the thyroid nuclear receptors, most of scientists have focused on the effects of transcriptional changes of thyroid hormones.

T3 hormone binds very efficiently to the nuclear receptors, whereas the T4 hormone binds less efficiently. The hormones derived from T4 and T3 do not bind to the nuclear receptors (Koerner et al., J. Biol. Chem., 1975; Lazar, Endocrine Rev., 1993; Hulbert, Bio. Rev., 2000; Oppenheimer, Biochimie, 1999; Yen, Physiol. Rev., 2001).

The use of T3 hormone for treating obesity is well known by the man skilled in the art. However, its use has been highly limited because of serious side effects of T3 hormone, particularly cardiac side effects. The treatment of hypothyroidism lies on T3, which can be used directly or produced in vivo by the transformation of its very little active precursor, the T4 hormone (Yen, Physiol. Rev., 2001). T3 is known as the real active thyroid, hormone.

The effects induced by thyroid hormones, such as T3, via the nuclear receptor pathway are physiologically important effects observed at very low concentrations. These effects are often deleterious when T3 is administered to subjects that do not suffer from hypothyroidism. These effects can be considered as “hyperthyroidic effects” linked to the nuclear receptor pathway.

The international application WO2005/009433 and the corresponding scientific paper (Lanni et al., The FASEB Journal, 2005) have disclosed an effect of the 3,5-T2 on energetic metabolism. More particularly, normal rats receiving a high-fat diet and treated with a daily peritoneal injection of 3,5-T2 gained less weight and had less fat deposit than untreated rats. The 3,5-T2 hormone was thus proposed for the treatment of obesity and related pathologies.

Obesity is one of the major public health concerns in developed countries as well as in developing countries. The mechanisms involved in obesity are not really understood. Factors involved in obesity are particularly alimentation (fat and sweet diets) and environment conditions (physical activity, social environment, food availability).

In prior art, it has never been disclosed that thyroid hormones may have effects on insulin and glycemia.

Diabetes is a chronic disease characterized by a hyperglycemia.

Type 1 diabetes results from the destruction of the pancreatic β cells secreting insulin. Treatment of type 1 diabetes particularly consists in the administering of insulin.

Type 2 diabetes is more frequent than type 1 diabetes in the population and is generally associated to obesity. Type 2 diabetes is characterized by two interdependent abnormalities: an insulino-resistance and a reduced production of insulin by response to glycemia.

Treatments of type 2 diabetes particularly consist in using an agonist drug of insulin or an agonist of insulin secretion by the beta cells, in reducing the glycemia and the weight of the diabetic patients.

Besides, the administration modes classically used for the treatment of diabetes, obesity and related pathologies resort to galenic formulations which might involve the possible degradation of the active substances, in particular thyroid hormones, by liver.

More efficient and more appropriate treatments (particularly in term of side effects, comfort of patients such as frequency of use and administering route) are needed against chronic diseases such as diabetes, obesity and dyslipidemia.

One aim of the invention is to provide a new therapeutic class of drugs for the treatment of diabetes.

Another aim of the invention is to provide a combination product for a simultaneous, separate or sequential use intended for the treatment of diabetes.

Another aim of the present invention is to provide new pharmaceutical compositions comprising a thyroid hormone as active substance, the galenic formulation of which is such that the active substances can be used in reduced amounts compared to those commonly used in the prior art.

Another aim of the present invention is to provide new pharmaceutical compositions comprising a thyroid hormone as active substance for the treatment of diabetes, obesity and related pathologies.

The present invention relates to the use of at least one hormone chosen among 3,5-diiodothyronine, 3′,3-diiodothyronine, 3′,5-diiodothyronine, 3′-iodothyronine, 3-iodothyronine or 5-iodothyronine, for the preparation of a drug intended for the treatment of pathologies chosen among hyperglycemia, insulin resistance, beta pancreatic cell insufficiency or related pathologies.

According to the present invention, the terms “3,5-diiodothyronine, 3′,3-diiodothyronine, 3′,5-diiodothyronine, 3′-iodothyronine, 3-iodothyronine and 5-iodothyronine” refer respectively to 3,5-T2, 3′,3-T2, 3′,5-T2, 3′-T, 3-T and 5-T.

The Inventors have shown for the first time that 3,5-T2, 3′,3-T2, 3′,5-T2,3′-T, 3-T and 5-T are capable of reducing glycemia and insulin plasmatic concentrations. These thyroid hormones can therefore be used for the treatment of pathologies chosen among hyperglycemia, insulin resistance, beta pancreatic cell insufficiency or related pathologies.

Furthermore, the Inventors propose that 3,5-T2 has beneficial effect only on the glycemia of diabetic subjects and has no significant effect on glycemia of non diabetic subjects (see Examples section).

Hyperglycemia is characterized by fasting glucose concentrations higher than 1 g/l (or 100 mg/dl or 5.5 mmol/l), particularly higher than 1.2 g/l. The use of 3,5-T2, 3′,3-T2, 3′,5-T2, 3′-T, 3-T and 5-T allows reducing glycemia to normal concentrations.

By the expression “normal concentrations of glucose”, one means glucose plasmatic concentration comprised from 4.4 mmol/l to 5.5 mmol/l, “abnormal” blood glucose is defined by fasting plasma glucose >5.55 mmol/l and diabetes by fasting plasma glucose >6.1 mmol/l (Meggs et al., Diabetes, 2003).

Glycemia is assessed by classical blood tests using the glucose oxidase method as reference (Yeni-Komshian et al., Diabetes Care, 2000, p 171-175; Chew et al., MJA, 2006, p 445-449; Wallace et al., Diabetes Care, 2004, p 1487-1495).

Insulin resistance is characterized by insulin plasmatic concentrations higher than 8 mU/l or 60 pmol/l (Wallace et al., Diabetes Care, 2004, p 1487-1495).

Insulin resistance is the condition in which normal amounts of insulin are inadequate to produce a normal response from fat, muscle and liver cells, i.e. a resistance to the physiological action of insulin.

It is defined as the lowest quartile of measures of insulin sensitivity (e.g. insulin stimulated glucose uptake during euglycaemic clamp) or highest quartile of fasting insulin or homeostasis model assessment (HOMA) insulin resistance index in the population studied (Alberti et al. “Definition, diagnosis and classification of diabetes mellitus and its complications. Part 1: Diagnosis and classification of diabetes mellitus, provisional report of a WHO consultation”, Diabetic Med, 1998, p 539-553; Wallace et al., Diabetes Care, 2004, p 1487-1495).

The use of the above-mentioned active substances allows reducing insulin plasmatic concentrations to normal concentrations, increasing the sensitivity to insulin and improving the metabolism of glucose and lipids.

By the expression “normal concentrations of insulin”, one means insulin plasmatic concentration comprised, from 5 to 8 mU/l (36 to 60 pmol/l).

Insulin concentration is assessed by classical blood tests (RIA assay with human antibody; Yeni-Komshian et al., Diabetes Care, 2000, p 171-175; Chew et al., MJA, 2006, p 445-449; Wallace et al., Diabetes Care, 2004, p 1487-1495).

Sensitivity to insulin can be assessed by the HOMA (Homeostasis Model Assessment) method (Wallace et al., Diabetes Care, 2004, p 1487-1495, see FIG. 2 on page 1489).

Surprisingly, the use of the above-mentioned active substances seems to improve pancreatic β cells survival, and thus the regeneration of said insulin secreting cells.

The regeneration of said cells is evaluated through the measurement of insulin concentration (RIA assay with human antibody; Yeni-Komshian et al., Diabetes Care, 2000, p 171-175; Chew et al., MJA, 2006, p 445-449; Wallace et al., Diabetes Care, 2004, p 1487-1495).

Results obtained on ZDF rats show that treatment with 3,5-T2 induced decreasing glucose concentration and increasing plasmatic insulin concentration.

In Goto-Kakizaki (GK) rats, a genetic model of type 2 diabetes, there is a restriction of the β cell mass as early as fetal age, which is maintained in the adult animal The restriction of the β cell mass can be considered as a crucial event in the sequence leading to overt diabetes in this model In the GK model, the regeneration of β cells occurs with a lower efficiency as compared to non-diabetic Wistar rats. This defect in the GK rats is both the result of genetic predisposition contributing to an altered β cells neogenesis potential and environment factors, such as chronic hyperglycemia, leading to a reduced β cell proliferative capacity specific to the adult animals. These results are described in Movassat et al. Diabetologia, 1997, p 916-925 and in Plachot et al., Histochem Cell Biol, 2001, p 131-139, the entire contents of which are incorporated herein by reference.

Assuming that a chronic hyperglycemia induced, a destruction of pancreatic β cells and thus a decreased secretion of insulin, restored normal insulin concentrations could, mean that the β cells are regenerated.

The β cells functional mass can be correlated to the level of insulin secretion through the HOMA method. On animal models, the man skilled in the art can envision the direct evaluation of pancreas mass.

The present invention particularly relates to the use as defined above, wherein said hormone is chosen among 3,5-diiodothyronine, 3′,3-diiodothyronine or 3′,5-diiodothyronine.

The present invention further relates to the use as defined above, for the treatment of diabetes, particularly type 1 or 2 diabetes.

If the above-mentioned hormones are administrated, in classical dosages and in galenic formulations classically used in the prior art, the treated pathologies exclude:
pre-pathologic and pathologic states related to overweight, obesity, alcoholic and non-alcoholic hepatic steatosis, dyslipidemia including hypercholesterolemia and hypertriglyceridemia, atherosclerosis, hepatopathies associated to a dysmetabolism, altered lipid metabolism in diabetic subjects, cholecistopathies, deposition of subcutaneous fat including cellulite, vasomotor rhinitis including the allergic one,
skin disorders including stria, cellulite, roughened skin, actinic skin damage, intrinsically aged skin, photodamaged skin, lichen planus, ichthyosis, acne, psoriasis, wrinkled skin, Dernier's disease, eczema, atopic dermatitis, seborrheic dermatitis scleroderma, collagen deficient skin, glucocorticoid induced atrophy, chloracne, pityriasis, clogging of sebaceous epithelium, disturbances of keratinization, acne rosacea, xanthoma, dry scaling dermatitides, alopecia, erythema, senile eczema, keratosispilaris, acute seborrhoea, skin scarring, preventive treatment prior to cosmetic surgery.

The present invention also relates to a pharmaceutical composition comprising as active substance at least one hormone chosen among 3,5-diiodothyronine, 3′,3-diiodothyronine, 3′,5-diiodothyronine, 3′-iodothyronine, 3-iodothyronine or 5-iodothyronine, in association with a pharmaceutically acceptable vehicle suitable for an administration via a subcutaneous or transcutaneous route.

By the expression “pharmaceutically acceptable vehicle”, one means pharmaceutically acceptable solid or liquid, diluting or encapsulating, filling or carrying agents, which are usually employed in pharmaceutical industry for making pharmaceutical compositions.

In the subcutaneous route, the drag can be injected directly into fatty tissue just beneath the skin or the drug can be included, in capsules that are inserted under the skin.

In the transcutaneous route, the drug passes through the skin to the bloodstream without injection. Particularly, the drug is comprised in a patch applied on the skin. Concerning patches formulation, the drag can be mixed with a chemical, such as alcohol, to enhance skin penetration.

The dosage forms include immediate release, extended release, pulse release, variable release, controlled release, timed release, sustained release, delayed release, long acting, and combinations thereof.

In an advantageous embodiment of the invention, the pharmaceutical composition is suitable for a transcutaneous, particularly by the means of patches.

In an advantageous embodiment, the administration of the pharmaceutical composition avoids partially that the drug passes through liver, which is susceptible of an important degradation of the hormones.

In another advantageous embodiment of the invention, the pharmaceutical composition is suitable for a subcutaneous administration, particularly by the means of a capsule injected beneath the skin.

In another advantageous embodiment of the invention, the pharmaceutical composition is suitable for the treatment of all pathologies, in particular the pathologies chosen among:
hyperglycemia, insulin resistance, beta pancreatic cell insufficiency, diabetes, or related pathologies,
obesity, overweight or related pathologies, hypercholesterolemia, hypertriglyceridemia, dyslipidemia, alcoholic and non alcoholic hepatic steatosis, atherosclerosis, hepatopathies associated to a dysmetabolism, cholecystopathies, deposit of subcutaneous fat, particularly cellulite or vasomotor rhinitis.

The present invention further relates to pharmaceutical composition as defined above, wherein said pharmaceutically acceptable vehicle allows a continuous, preferably constant, release, of said active substance.

The continuous, preferably constant, release of the active substance allows obtaining:
increased effects on metabolic disorders as compared to results obtained via another administration mode, or
newly observed effects on metabolic disorders on animal models on which there were previously no positive results.

By the expression “continuous release”, one means a continuous release of the drug over at least 24 hours, preferably at least one month, most preferably at least two months, in particular three months.

By the expression “constant release”, one means a continuous release of the drug over at least 24 hours, preferably at least one month, most preferably at least two months, in particular three months, the quantity of released drug/time unit being essentially constant.

A continuous and constant release is for example achieved by using patches or capsules injected under the skin.

In an advantageous embodiment of the invention, the pharmaceutical composition is suitable for the treatment of all pathologies, in particular the pathologies chosen among:
hyperglycemia, insulin resistance, beta pancreatic cell insufficiency, diabetes, or related pathologies,
obesity, overweight or related pathologies, hypercholesterolemia, hypertriglyceridemia, dyslipidemia, alcoholic and non alcoholic hepatic steatosis, atherosclerosis, hepatopathies associated to a dysmetabolism, cholecystopathies, deposit of subcutaneous fat, particularly cellulite or vasomotor rhinitis.

The present invention particularly relates to a pharmaceutical composition as defined above, in a suitable form for the release of about 0.01 μg/kg/day to about 250 μg/kg/day, particularly about 0.01 μg/kg/day to about 25 μg/kg/day, particularly about 0.1 μg/kg/day to about 15 μg/kg/day of active substance, more particularly about 0.1 μg/kg/day to about 5 μg/kg/day of active substance, most particularly about 0.1 μg/kg/day to 1 μg/kg/day of active substance.

The dosage of active substance particularly depends on the administration route, which is easily determined by the man skilled in the art.

In an advantageous embodiment of the invention, the pharmaceutical composition is suitable for the treatment of all pathologies, in particular the pathologies chosen among:
hyperglycemia, insulin resistance, beta pancreatic cell insufficiency, diabetes, or related pathologies,
obesity, overweight or related pathologies, hypercholesterolemia, hypertriglyceridemia, dyslipidemia, alcoholic and non alcoholic hepatic steatosis, atherosclerosis, hepatopathies associated to a dysmetabolism, cholecystopathies, deposit of subcutaneous fat, particularly cellulite or vasomotor rhinitis.

The present invention further relates to pharmaceutical composition as defined above, comprising by dosage unit about 5 μg to about 1.5 g of active substance, particularly about 75 mg to about 750 mg of active substance, to be released in a lapse of time corresponding to the above-mentioned values of the ranges in μg/kg/day or mg/kg/day for a 70 kg human.

As an example, for the treatment of a 70 kg human, the dosage will be:
about 5 μg to about 150 mg, particularly about 5 μg to about 15 mg, particularly about 50 μg to about 10 mg, particularly about 50 μg to about 3 mg, most particularly about 50 μg to about 500 μg of active substance to achieve an eight day treatment,
about 20 μg to about 500 mg, particularly about 20 μg to about 50 mg, particularly about 200 μg to about 30 mg, particularly about 200 μg to about 10 mg, most particularly about 200 μg to about 2 mg of active substance to achieve a thirty day treatment,
about 60 μg to about 1.5 g, particularly about 60 μg to about 150 mg, particularly about 600 μg to about 100 mg, particularly about 600 μg to about 30 mg, most particularly about 600 μg to about 6 mg of active substance to achieve a ninety day treatment.

By the expression “dosage unit”, one means the quantity of active substance comprised in one drug unit.

Depending on the administration route and on the formulation of the pharmaceutical composition, the active substance comprised, in the dosage unit can be released quickly or continuously over a period of time. The pharmaceutical composition can also be a slow-release drug.

Pharmaceutical compositions of the invention may be administered in a partial dose or a dose one or more times during a 24 hour period. Fractional, double or other multiple doses may be taken simultaneously or at different times during a 24 hour period.

In an advantageous embodiment, the pharmaceutical composition of the invention is administered, in a unique dose, which allows a continuous release for a period of time of at least 24 h, preferably at least one week, more preferably at least one month, most preferably at least two months, in particular at least three months.

In another advantageous embodiment of the invention, the pharmaceutical composition is suitable for the treatment of all pathologies, in particular the pathologies chosen among:
hyperglycemia, insulin resistance, beta pancreatic cell insufficiency, diabetes, or related pathologies,
obesity, overweight or related pathologies, hypercholesterolemia, hypertriglyceridemia, dyslipidemia, alcoholic and non alcoholic hepatic steatosis, atherosclerosis, hepatopathies associated to a dysmetabolism, cholecystopathies, deposit of subcutaneous fat, particularly cellulite or vasomotor rhinitis.

The present invention further relates to a pharmaceutical composition as defined above, wherein said pharmaceutically acceptable vehicle is a chemical, such as alcohol, used to enhance skin penetration.

The means that allow a continuous and/or a constant release of the active substance are chosen among patches or capsules injected under the skin.

The present invention also relates to the use of at least one hormone chosen among 3,5-diiodothyronine, 3′,3-diiodothyronine, 3′,5-iodothyronine, 3′-iodothyronine, 5′-iodothyronine, 3-iodothyronine or 5-iodothyronine, for the preparation of a drug intended for the treatment of pathologies chosen among:
hyperglycemia, insulin resistance, beta pancreatic cell insufficiency, diabetes, or related pathologies,
obesity, overweight or related pathologies, hypercholesterolemia, hypertriglyceridemia, dyslipidemia, alcoholic and non alcoholic hepatic steatosis, atherosclerosis, hepatopathies associated to a dysmetabolism, cholecystopathies, deposit of subcutaneous fat, particularly cellulite or vasomotor rhinitis,
said hormone and said pharmaceutically acceptable vehicle being under a suitable form for an administration via a subcutaneous or transcutaneous route.

The present invention relates more particularly to the use as defined above, for the treatment of for the treatment of hyperglycemia, insulin resistance, beta pancreatic cell insufficiency or related pathologies, said hormone and said pharmaceutically acceptable vehicle being under a suitable form for an administration via a subcutaneous or transcutaneous route.

In an advantageous embodiment, the present invention relates to the use as defined above, for the treatment of diabetes, particularly type 1 or 2 diabetes, said hormone and said pharmaceutically acceptable vehicle being under a suitable form for an administration via a subcutaneous or transcutaneous route.

The present invention relates more particularly to the use as defined above for the treatment of pathologies chosen among:
hyperglycemia, insulin resistance, beta pancreatic cell insufficiency, diabetes, or related pathologies,
obesity, overweight or related pathologies, hypercholesterolemia, hypertriglyceridemia, dyslipidemia, alcoholic and non alcoholic hepatic steatosis, atherosclerosis, hepatopathies associated to a dysmetabolism, cholecystopathies, deposit of subcutaneous fat, particularly cellulite or vasomotor rhinitis,

wherein said pharmaceutically acceptable vehicle allows a continuous, preferably constant release of said active substance.

The present invention also relates more particularly to the use as defined above the treatment of pathologies chosen among:
hyperglycemia, insulin resistance, beta pancreatic cell insufficiency, diabetes, or related pathologies,
obesity, overweight or related pathologies, hypercholesterolemia, hypertriglyceridemia, dyslipidemia, alcoholic and non alcoholic hepatic steatosis, atherosclerosis, hepatopathies associated to a dysmetabolism, cholecystopathies, deposit of subcutaneous fat, particularly cellulite or vasomotor rhinitis,

wherein said hormone and said pharmaceutically acceptable vehicle are in a suitable form for in a suitable form for the release of about 0.01 μg/kg/day to about 250 μg/kg/day, particularly about 0.01 μg/kg/day to about 25 μg/kg/day, particularly about 0.1 μg/kg/day to about 15 μg/kg/day of active substance, more particularly about 0.1 μg/kg/day to about 5 μg/kg/day of active substance, most particularly about 0.1 μg/kg/day to 1 μg/kg/day of active substance.

The present invention also relates to a product comprising:
at least one hormone chosen among 3,5-diiodothyronine, 3′,3-diiodothyronine, 3′,5-diiodothyronine, 3′-iodothyronine, 3-iodothyronine or 5-iodothyronine, and
at least one active substance activating the pancreatic secretion of insulin, particularly chosen among antidiabetic oral drags, or susceptible of slowing the digestive absorption of glucose,

as a combination product for a simultaneous, separated or sequential use intended for the treatment of diabetes.

The present invention also relates to nutraceutics or food compositions comprising at least one hormone chosen among 3,5-diiodothyronine, 3′,3-diiodothyronine, 3′,5-diiodothyronine, 3′-iodothyronine, 3-iodothyronine or 5-iodothyronine.

The present invention also relates to a method for improving meat quality of mammals and birds, in particular pork and beef meat quality, by controlling the ratio between the weight of adipose tissues and lean tissues, in particular by:
lowering the weight of adipose tissues in animals as compared to the weight of adipose tissues of animals fed with a normal diet, and
maintaining or increasing the weight of lean tissues as compared to the weight of lean tissues of animals fed with a normal diet,

by the administration of nutraceutics or food compositions comprising at least one hormone chosen among 3,5-diiodothyronine, 3′,3-diiodothyronine, 3′,5-diiodothyronine, 3′-iodothyronine, 3-iodothyronine or 5-iodothyronine.

### DRAWINGS

FIGS. 1A, 1B and 1C

Growth rate of Wistar rats treated with a high dosage of 3,5-T2 (25 μg/100 g of body weight (BW)), a low dosage of 3,5-T2 (2.5 μg/100 g BW) or a high dosage of 3,3′-T2 (25 μg/100 g BW).

FIGS. 1A, 1B and 1C represent the weight of the rats (in grams) relative to time (in days) for a period of 20 or 35 days. The weight of the rats treated with thyroid hormones is shown on the curve with white rectangles and the weight of those treated with placebo is represented with black rectangles (FIG. 1A) or black diamonds (FIGS. 1B and 1C).

FIG. 1A: the rats were treated with a high dosage of 3,5-T2.

FIG. 1B: the rats were treated with a low dosage of 3,5-T2.

FIG. 1C: the rats were treated with a high dosage of 3,3′-T2.

FIGS. 2A, 2B and 2C

Food intake of Wistar rats treated with a high dosage of 3,5-T2 (25 μg/100 g BW), a low dosage of 3,5-T2 (2.5 μg/100 g BW) or a high dosage of 3,3′-T2 (25 μg/100 g BW).

FIGS. 2A, 2B and 2C represent the food intake in grams/day of the rats relative to time (in days) for a period of 21, 24 or 32 days. The food intake of the rats treated with thyroid hormones is shown on the curve with white rectangles and the food intake of those treated with placebo is represented with black diamonds.

FIG. 2A: the rats were treated with a high dosage of 3,5-T2,

FIG. 2B: the rats were treated with a low dosage of 3,5-T2.

FIG. 2C: the rats were treated with a high dosage of 3,3′-T2.

FIGS. 3A and 3B

Energy expenditure of Wistar rats treated with a low dosage of 3,5-T2 (2.5 μg/100 g BW) or a high dosage of 3,3′-T2 (25 μg/100 g BW).

FIGS. 3A and 3B represent the energy expenditure (EE) in Kcal/day/kg^{0.75} of the rats relative to time (in minutes). The energy expenditure of the rats treated with thyroid hormones is shown on the curve with white circles (FIG. 3A) or white diamonds (FIG. 3B) and the energy expenditure of those treated with placebo is represented with black circles.

The horizontal black line indicates a period where the rats are in the dark.

FIG. 3A: the rats were treated with a low dosage of 3,5-T2.

FIG. 3B: the rats were treated with a high dosage of 3,3′-T2.

FIGS. 4A and 4B

Respiratory quotient (RQ) of Wistar rats treated with a low dosage of 3,5-T2 (2.5 μg/100 g BW) or a high dosage of 3,3′-T2 (25 μg/100 g BW).

FIGS. 4A and 4B represent the respiratory quotient of the rats relative to time (in minutes). The respiratory quotient of the rats treated with thyroid hormones is shown on the curve with white circles (FIG. 4A) or white diamonds (FIG. 4B) and the respiratory quotient of those treated with placebo is represented with black circles.

The horizontal black line indicates a period where the rats are in the dark.

FIG. 4A: the rats were treated with a low dosage of 3,5-T2.

FIG. 4B: the rats were treated with a high dosage of 3,3′-T2.

FIGS. 5A, 5B and 5C

Weight of adipose tissues, skeletal muscles and brown adipose tissue of Wistar rats treated with a high dosage of 3,5-T2 (25 μg/100 g BW).

The results of the rats treated, with thyroid hormone are shown in white and the results of those treated with placebo in black. The left column gives the weight in grams and the right column the relative weight in g/100 g of body weight.

The asterisk corresponds to a p-value <0.01.

FIG. 5A: the upper panel gives the weight (g) of different adipose tissues (retroperitoneal, epididymal, mesenteric and subcutaneous fat) and the lower panel gives the relative weight (g/100 g BW) of these adipose tissues.

FIG. 5B: the left panel gives the weight (mg) of skeletal muscles (soleus and plantaris muscles) and the right panel gives the relative weight (mg/100 g BW) of these muscles.

FIG. 5C: the left panel gives the weight (g) of interscapular brown adipose tissue and the right panel gives the relative weight (g/100 g BW) of this tissue.

FIGS. 6A, 6 B and 6C

Weight of adipose tissues, skeletal muscles and brown adipose tissue of Wistar rats treated with a low dosage of 3,5-T2 (2.5 μg/100 g BW).

The results of the rats treated, with thyroid hormone are shown in white and the results of those treated with placebo in black. The left column gives the weight in grams and the right column the relative weight in g/100 g of body weight.

The asterisk corresponds to a p-value <0.01.

FIG. 6A: the upper panel gives the weight (g) of different adipose tissues (retroperitoneal, epididymal, mesenteric and subcutaneous fat) and the lower panel gives the relative weight (g/100 g BW) of these adipose tissues.

FIG. 6B: the left panel gives the weight (g) of skeletal muscles (soleus and plantaris muscles) and the right panel gives the relative weight (mg/100 g BW) of these muscles.

FIG. 6C: the left panel gives the weight (g) of interscapular brown adipose tissue and the right panel gives the relative weight (g/100 g BW) of this tissue.

FIGS. 7A, 7B, 7C and 7D

Weight of adipose tissues, skeletal muscles and brown adipose tissue of Wistar rats treated with a high dosage of 3,3′-T2 (25 μg/100 g BW).

The results of the rats treated with thyroid hormone are shown in white and the results of those treated with placebo in black. The left column gives the weight in grams and the right column the relative weight in g/100 g of body weight.

The asterisk corresponds to a p-value <0.01.

FIG. 7A: the upper panel gives the weight (g) of different adipose tissues (retroperitoneal, epididymal, mesenteric and subcutaneous fat) and the lower panel gives the relative weight (g/100 g BW) of these adipose tissues.

FIG. 7B: the left panel gives the weight (g) of skeletal muscles (soleus and plantaris muscles) and the right panel gives the relative weight (mg/100 g BW) of these muscles.

FIG. 7C: the left panel gives the weight (g) of interscapular brown adipose tissue and the right panel gives the relative weight (g/100 g BW) of this tissue.

FIGS. 8A, 8B and 8C

Body weight and food intake of Zucker rats treated with a high dosage of 3,5-T2 (25 μg/100 g BW).

FIGS. 8A and 8B represent respectively the body weight in grams and the food, intake in grams/day of the rats relative to time (in days) for a period of 30 days.

The body weight and the food intake of the rats treated with thyroid hormone are shown on the curve with white rectangles and the body weight and the food intake of those treated with placebo are represented with black diamonds.

FIG. 8C is a photograph of two Zucker rats.

FIG. 8A: body weight (g).

FIG. 8B: food intake (g/day).

FIG. 8C: the rat on the top of the photograph is treated with placebo and the rat on the bottom of the photograph is treated with high dosage 3,5-T2.

FIGS. 9A, 9B and 9C

Weight of adipose tissues, skeletal muscles and brown adipose tissue of Wistar rats treated with a high dosage of 3,5-T2 (25 μg/100 g BW).

The results of the rats treated with thyroid hormone are shown in white and the results of those treated, with placebo in black. The left column gives the weight in grams and the right column the relative weight in g/100 g of body weight.

The asterisk corresponds to a p-value <0.01.

FIG. 9A: the upper panel gives the weight (g) of different adipose tissues (retroperitoneal, epididymal, mesenteric and subcutaneous fat) and the lower panel gives the relative weight (g/100 g BW) of these adipose tissues.

FIG. 9B: the left panel gives the weight (g) of skeletal muscles (soleus and plantaris muscles) and the right panel gives the relative weight (mg/100 g BW) of these muscles.

FIG. 9C: the left panel gives the weight (g) of interscapular brown adipose tissue and the right panel gives the relative weight (g/100 g BW) of this tissue.

FIGS. 10A, 10B, 10C and 10D

Body weight, weight of adipose tissues and weight of lean tissues and core temperature in Zucker Diabetic fatty (ZDF) rats treated with high dosage 3,5-T2 (25 μg/100 g BW).

FIG. 10A represents the body weight in grams of the rats relative to time (in days) for a period of 30 days.

The body weight of rats treated with thyroid hormone is shown on the curve with black rectangles and the body weight of those treated with placebo is represented, with white rectangles.

FIGS. 10B and 10C represent respectively the weight of adipose tissues and the weight of lean tissues of rats treated with a high dosage of 3,5-T2 or with placebo for a period of 4 weeks.

The basal values are shown in white and the values measured after 4 weeks in black.

FIG. 10D represents the core temperature (° C.) of rats treated with a high dosage of 3,5-T2, measured at different dates for a period of 15 days.

The core temperature of rats treated with thyroid hormone is shown in blade and the core temperature of those treated with placebo in white.

FIG. 10A: body weight (g).

FIG. 10B: weight of adipose tissues (g).

FIG. 10C: weight of lean tissues (g).

FIG. 10D: core temperature (° C.).

FIGS. 11A, 11B, 11C et 11D

Blood glucose concentrations, HbA1c percent, plasmatic concentrations of insulin and cholesterol and triglycerides in Zucker Diabetic fatty (ZDF) rats treated with a high dosage of 3,5-T2 (25 μg/100 BW).

The asterisk represents a p-value<0.01 and the triple asterisk a p-value<0.001.

FIG. 11A represents the plasmatic glucose concentration (mmol/l) in rats treated with a high dosage of 3,5-T2 for a period of 4 weeks.

The results of rats treated with thyroid hormone are shown in black and the results of those treated with placebo in white.

FIG. 11B the variations of HbA1c percent in rats treated with a high dosage of 3,5-T2 for a period of 4 weeks.

The HbA1c percent measured before the treatment is shown in white and the HbA1c percent measured after 4 weeks of treatment in black.

FIG. 11C represents the plasmatic concentrations of insulin (pmol/l) in rats treated with a high dosage of 3,5-T2.

FIG. 11D represents the plasmatic concentrations of cholesterol and triglycerides (g/l) in rats treated with a high dosage of 3,5-T2.

The results of rats treated with thyroid hormone are shown in black and the results of those treated with placebo in white (FIGS. 11C and 11D).

FIG. 11A: glucose (mmol/l).

FIG. 11B: HbA1c (%).

FIG. 11C: insulin (pmol/l).

FIG. 11D: cholesterol (g/l) and triglycerides (g/l).

FIGS. 12A, 12B, 12C, 12D, 12E amid 12F

Rate of liver mitochondrial oxygen consumption (JO₂ in nmol of O₂/min/mg of protein) of Wistar rats treated with a high (25 μg/100 g BW) or a low dosage (2.5 μg/100 g BW) of thyroid hormones.

All measurements were performed using mitochondria (1.0 mg mitochondrial protein/ml) incubated with various substrates:
GM: glutamate/malate (5 mM/2.5 mM)
SR: succinate/rotenone (5 mM/5 μM),
GMS: glutamate/malate/succinate (5 mM/2.5 mM/5 mM),
Palm: palmitoyl carnitine (55 μM),
Octa: octanoyl carnitine (100 μM),
TMPD/AsC: TMPD/ascorbate (0.5 mM/0.5 mM), and
TMPD/AsC/DNP: TMPD/ascorbate/DNP (0.5 mM/0.5 mM/75 μM).

JO₂ was recorded in the presence of the substrate and following the addition of 1 mM ADP (adenosine diphosphate) (state 3).

The oligomycin was added to the mitochondrial suspension to determine the non-phosphorylating respiratory-rate (state 4).

Oxygen consumption of rats treated with thyroid hormones is shown in white, and oxygen consumption of those treated with placebo in black.

The asterisk corresponds to a p-value <0.01.

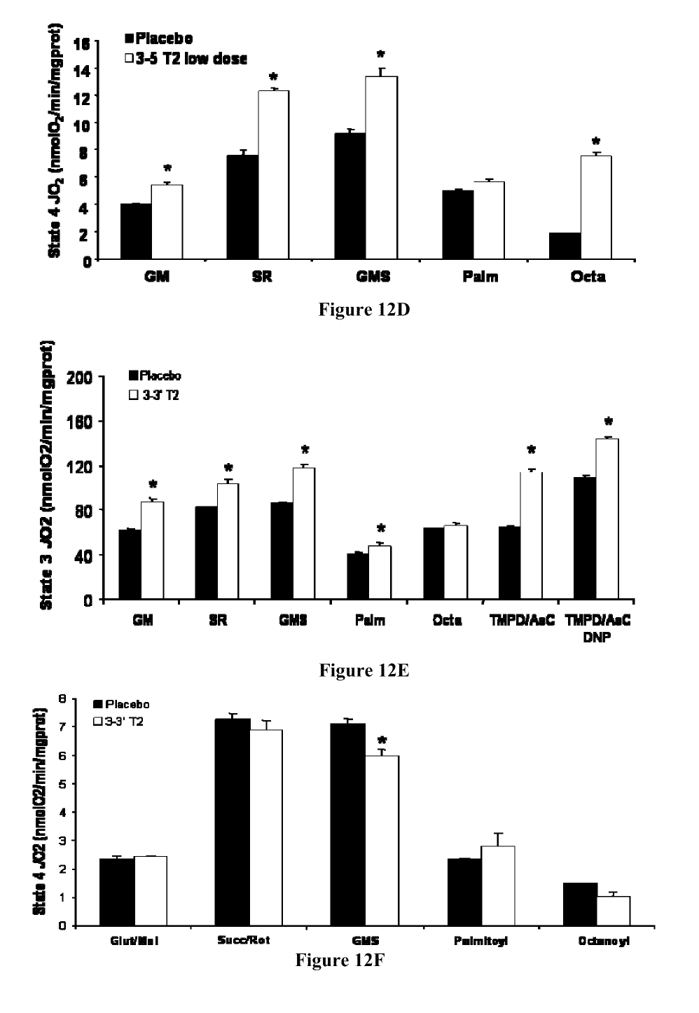

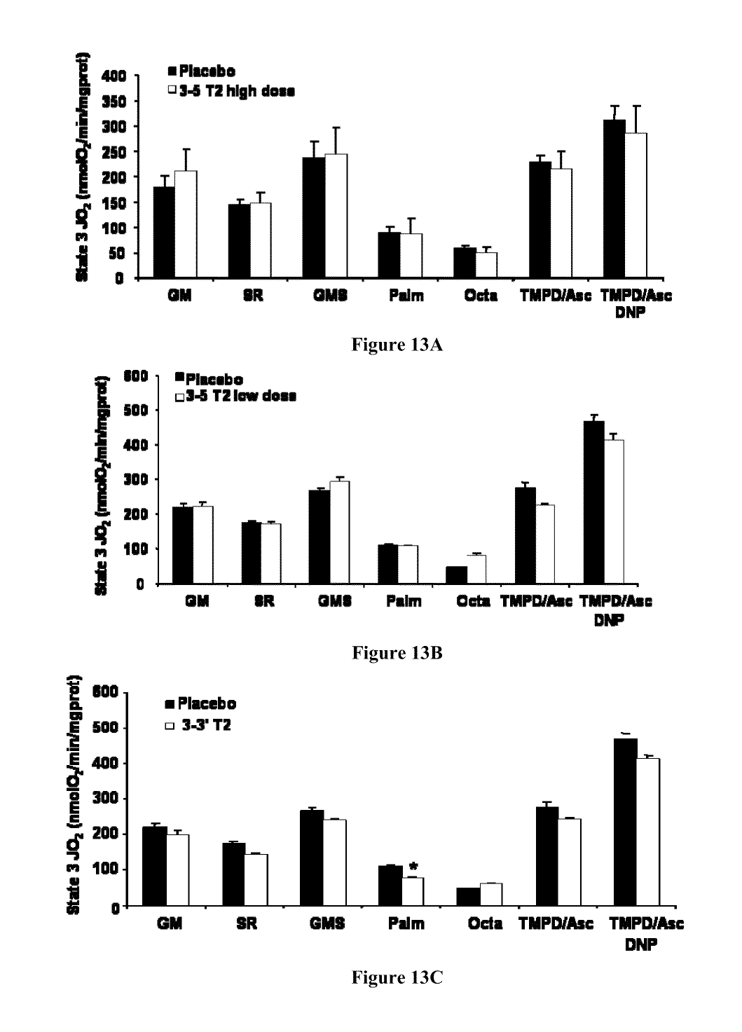

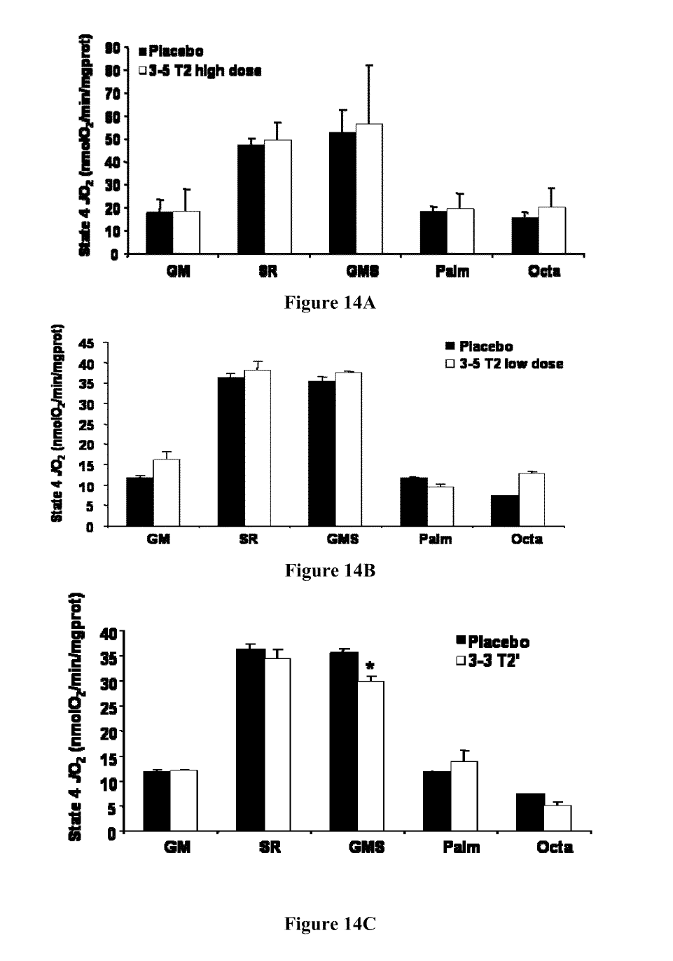

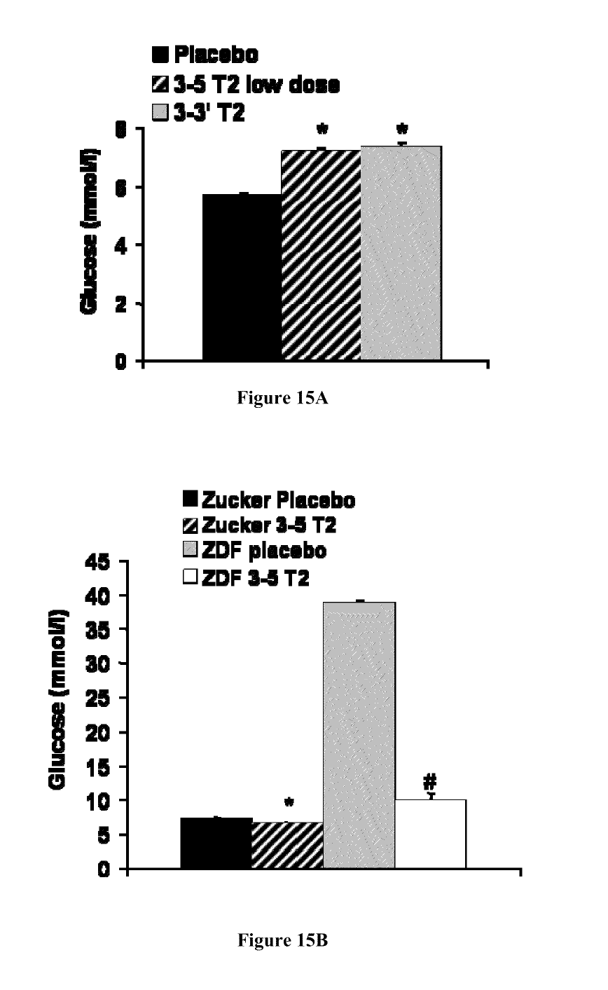

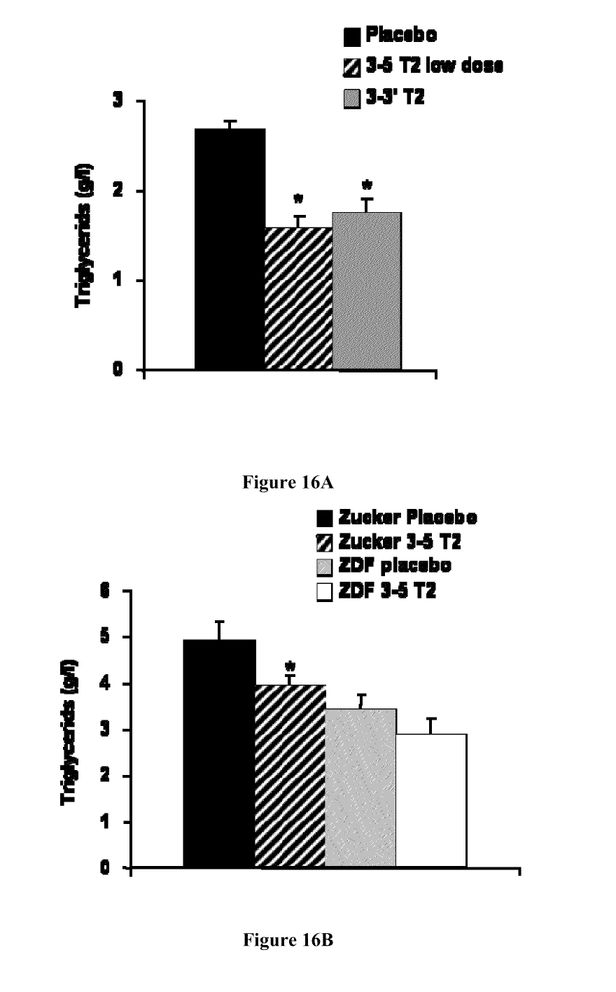

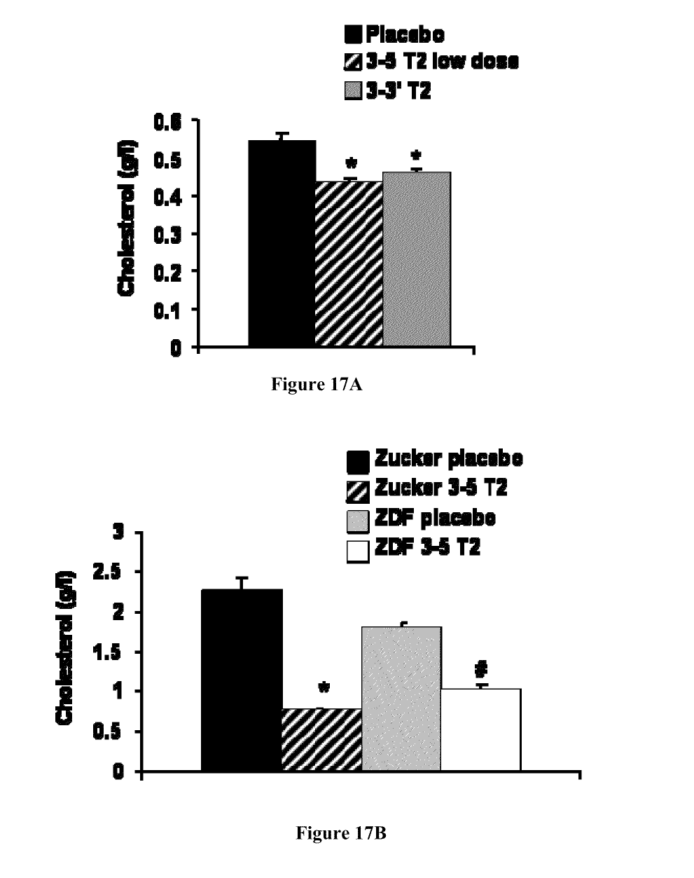

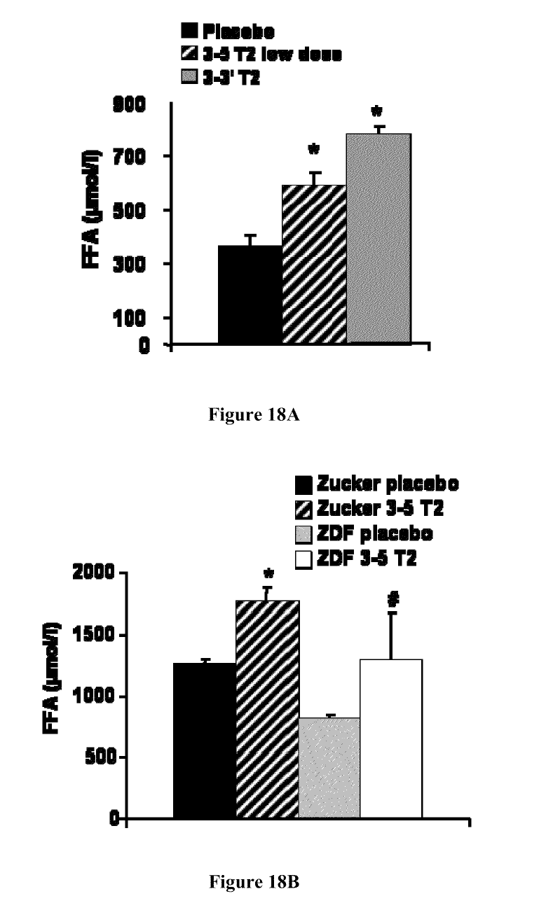

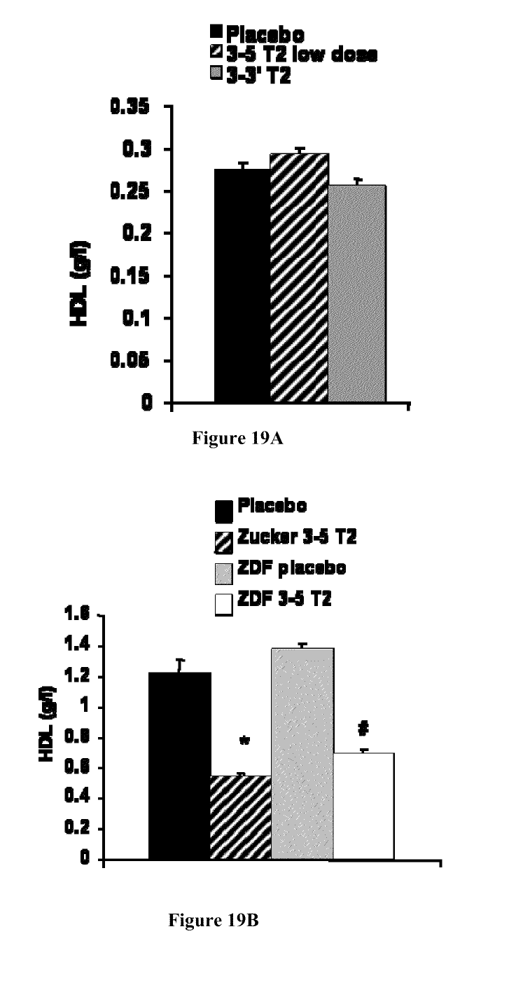

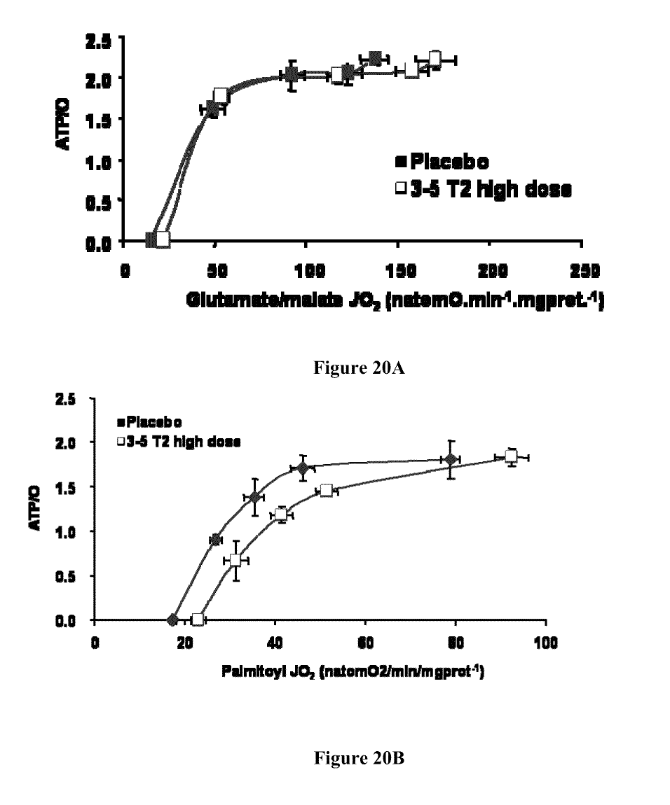

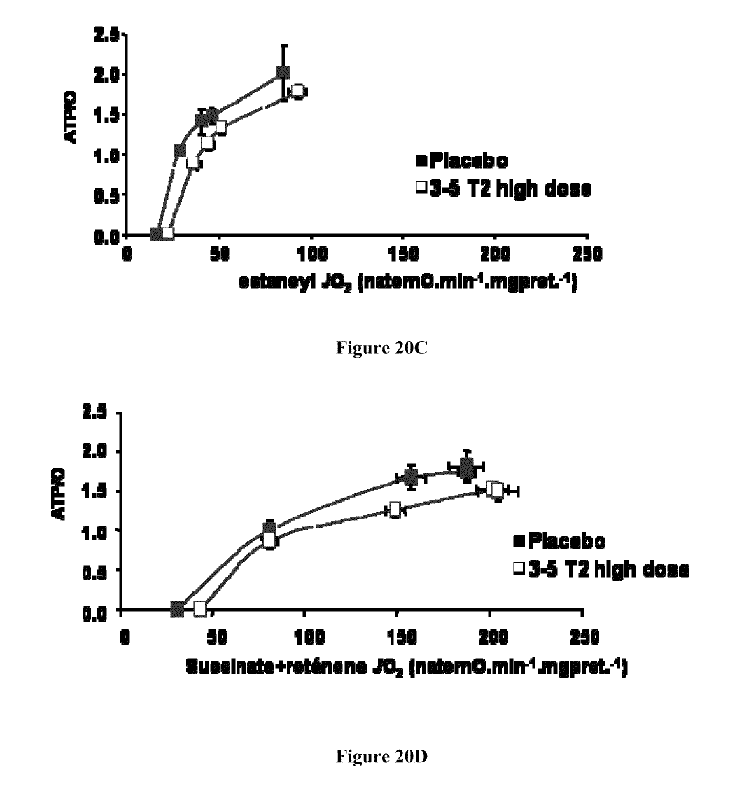

FIG. 12A: results obtained with rats treated with a high dosage of 3,5-T2 at state 3.

FIG. 12B: results obtained with rats treated with a high dosage of 3,5-T2 at state 4.

FIG. 12C: results obtained with rats treated with a low dosage of 3,5-T2 at state 3.

FIG. 12D: results obtained with rats treated, with a low dosage of 3,5-T2 at state 4.

FIG. 12E: results obtained, with rats treated with a high dosage of 3,3′-T2 at state 3.

FIG. 12F: results obtained with rats treated with a high dosage of 3,3′-T2 at state 4.

FIGS. 13A, 13B and 13C

Rate of muscle mitochondrial oxygen consumption (JO₂ in nmol of O₂/min/mg of protein) of Wistar rats treated with a high dosage (25 μg/100 g BW) or a low dosage (2.5 μg/100 g BW) of 3,5-T2 or a high dosage (25 μg/100 g BW) of 3,3′-T2.

All measurements were performed using mitochondria (0.2 mg mitochondrial protein/ml) incubated with various substrates:
GM: glutamate/malate (5 mM/2.5 mM)
SR: succinate/rotenone (5 mM/5 μM),
GMS: glutamate/malate/succinate (5 mM/2.5 mM/5 mM),
Palm: palmitoyl carnitine (55 μM),
Octa: octanoyl carnitine (100 μM),
TMPD/AsC: TMPD/ascorbate (0.5 mM/0.5 mM), and
TMPD/AsC/DNP: TMPD/ascorbate/DNP (0.5 mM/0.5 mM/75 μM).

JO₂ was recorded in the presence of the substrate and following the addition of 1 mM ADP (state 3).

The oligomycin was added to the mitochondrial suspension to determine the non-phosphorylating respiratory rate (state 4).

Oxygen consumption of rats treated with thyroid hormones is shown in white, and oxygen consumption of those treated with placebo in black.

The asterisk corresponds to a p-value <0.01.

FIG. 13A: results obtained with rats treated with high dosage of 3,5-T2 at state 3.

FIG. 13B: results obtained with rats treated with low dosage of 3,5-T2 at state 3.

FIG. 13C: results obtained with rats treated with high dosage of 3,3′-T2 at state 3.

FIGS. 14A, 14B and 14C

Rate of muscle mitochondrial oxygen consumption (JO₂ in nmol of O₂/min/mg of protein) of Wistar rats treated with a high dosage (25 μg/100 g BW) or a low dosage (2.5 μg/100 g BW) of 3,5-T2 or a high dosage (25 μg/100 g BW) of 3,3′-T2.

All measurements were performed using mitochondria (0.2 mg mitochondrial protein/ml) incubated with various substrates:
GM: glutamate/malate (5 mM/2.5 mM)
SR: succinate/rotenone (5 mM/5 μM),
GMS: glutamate/malate/succinate (5 mM/2.5 mM/5 mM),
Palm: palmitoyl carnitine (55 μM), and
Octa: octanoyl carnitine (100 μM).

JO₂ was recorded in the presence of the substrate and following the addition of 1 mM ADP (state 3).

The oligomycin was added to the mitochondrial suspension to determine the non-phosphorylating respiratory rate (state 4).

Oxygen consumption of rats treated with thyroid hormones is shown in white, and oxygen consumption of those treated with placebo in black.

The asterisk corresponds to a p-value <0.01.

FIG. 14A: results obtained with rats treated with high dosage of 3,5-T2 at state 4.

FIG. 14B: results obtained with rats treated with low dosage of 3,5-T2 at state 4.

FIG. 14C: results obtained with rats treated with high dosage of 3,3′-T2 at state 4.

FIGS. 15A and 15B

Plasmatic concentrations (mmol/l) of glucose in Wistar, Zucker and Zucker Diabetic fatty (ZDF) rats treated with thyroid, hormones.

These measurements were done on venous blood of the rats the day of the sacrifice.

The asterisk corresponds to a p-value <0.01 (vs Wistar and Zucker placebo) and the hash sign a p-value<01(vs ZDF placebo).

FIG. 15A: glucose (mmol/l) in Wistar rats treated with a low dosage of 3,5-T2 (2.5 μg/100 g BW), or 3,3′-T2.

FIG. 15B: glucose (mmol/l) in Zucker and ZDF rats treated with 3,5-T2 (25 μg/100 g BW).

FIGS. 16A and 16B

Plasmatic concentrations (g/l) of triglycerides in Wistar, Zucker and Zucker Diabetic fatty (ZDF) rats treated, with thyroid hormones.

These measurements were done on venous blood of the rats the day of the sacrifice (21^{st} day).

The asterisk corresponds to a p-value <0.1 (vs Wistar and Zucker placebo) and the hash sign a p-value<0.01 (vs ZDF placebo).

FIG. 16A: triglycerides (TG) (g/l) in Wistar rats treated with a low dosage of 3,5-T2 (2.5 μg/100 g BW), or 3,3′-T2.

FIG. 16B: triglycerides (TG) (g/l) in Zucker and ZDF rats treated with 3,5-T2 (25 μg/100 g BW).

FIGS. 17A and 17B

Plasmatic concentrations (g/l) of cholesterol in Wistar, Zucker and Zucker Diabetic fatty (ZDF) rats treated with thyroid, hormones.

These measurements were done on venous blood of the rats the day of the sacrifice.

The asterisk corresponds to a p-value <0.01 (vs Wistar and Zucker placebo) and the hash sign a p-value<0.01 (vs ZDF placebo).

FIG. 17A: cholesterol (g/l) in Wistar rats treated with a low dosage of 3,5-T2 (2.5 μg/100 g BW), or 3,3′-T2.

FIG. 17B: cholesterol (g/l) in Zucker and Zucker Diabetic fatty rats treated with 3,5-T2 (25 μg/100 g BW).

FIGS. 18A and 18B

Plasmatic concentrations (μmol/l) of FFA (Free Fatty Acid) in Wistar, Zucker and Zucker Diabetic fatty (ZDF) rats treated with thyroid hormones.

These measurements were done on venous blood of the rats the day of the sacrifice.

The asterisk corresponds to a p-value <0.01 (vs Wistar and Zucker placebo) and the hash sign a p-value<0.01 (vs ZDF placebo).

FIG. 18A: FFA (μmol/l) in Wistar rats treated with a low dosage of 3,5-T2 (2.5 μg/100 g BW), or 3,3′-T2,

FIG. 18B: FFA (μmol/l) in Zucker and ZDF rats treated with 3,5-T2 25 μg/100 g BW).

FIGS. 19A and 19B

Plasmatic concentrations (mmol/l) of HDL (Heavy Density Lipoprotein) in Wistar, Zucker and Zucker Diabetic fatty (ZDF) rats treated with thyroid hormones.

These measurements were done on venous blood of the rats the day of the sacrifice.

The asterisk corresponds to a p-value <0.01 (vs Wistar and Zucker placebo) and the hash sign a p-value<0.01 (vs ZDF placebo).

FIG. 19A: HDL (g/l) in Wistar rats treated with a low dosage of 3,5-T2 (2.5 μg/100 g BW, or 3,3′-T2.

FIG. 19B: HDL (g/l) in Zucker and ZDF rats treated with 3,5-T2 (25 μg/100 g BW).

FIGS. 20A, 20B, 20C and 20D

Effect of 3,5-T2 on oxidative phosphorylation efficiency investigated in liver mitochondria of Wistar rats isolated after 3 weeks of continuous treatment administered subcutaneously (250 μg/kg).

FIGS. 20A, 20B, 20C and 20D represent the ratio between ATP synthesis (nmol/min/g prot) and liver mitochondrial oxygen consumption (nmol/min/g prot) (P/O) as a function of liver mitochondrial oxygen consumption.

The P/O values of rats treated with 3,5-T2 are shown on the curve with white rectangles and the P/O values of those treated with placebo are represented with black rectangles (FIGS. 20A, 20C and 20D) or black diamonds (FIG. 20B).

All measurements were performed using mitochondria (1.0 mg mitochondrial protein/ml) incubated with various substrates:
GM: glutamate/malate (5 mM/2.5 mM)
Palm: palmitoyl carnitine (55 μM)
Octa: octanoyl carnitine (100 μM) and
Succ+Rot: succinate/rotenone (5 mM/5 μM)

FIG. 20A: P/O obtained, after incubation with GM substrate.

FIG. 20B: P/O obtained after incubation with Palm substrate.

FIG. 20C: P/O obtained after incubation with Octa substrate.

FIG. 20D: P/O obtained after incubation with Succ F Rot substrate.

### EXAMPLES

### Example 1

### Use of the 3,5-T2 Hormone for the Treatment of Obesity and Dyslipidemia

### 1. Material and Methods

Animal Handling

Adult male rat bred in the animal room facilities of Laboratory of Fundamental and Applied. Bioenergetics (Wistar strain) or purchased from Charles-River Laboratories, Domaine des oncins, L'ARBRESLE France [(genetically obese normoglycemic (Zucker or Fa/Fa) or diabetic (ZDF)] were caged individually in stainless steel hanging cages and maintained in a 22° C., 50±10% relative humidity and 12 h:12 h light:dark environment. All animals were fed ad libitum with a standard rat chow (Safe A04, Villemoisson, France) and tap water. Body mass and food intake were recorded twice/thrice a week and fresh food was provided, at the same time to ensure minimal disturbance to the animals' food, behavior.

Pellet Implant

Eight-week old rats (300 g±10 g) were anesthetized by simultaneous intraperitoneal injection of diazepam 4 mg/kg and ketamine 100 mg/kg. In order to maintain body temperature during the surgery (10 min), animals were placed on a warm blanket. After interscapular shaving, a small incision of 0.5 cm of the skin allows the subcutaneous implantation of a small pellet (containing rT3 or 3′,3-T2) with a 10-gauge precision trochar. The pellets, manufactured by Innovative Research of America (Sarasota, Fla., USA) are constituted of a biodegradable matrix that effectively and continuously release the active product in the animal.

3-5 diiodothyronine (3-5 T2) or 3-3′diiodothyronine (3-3′ T2) were used at different doses (5, 0.5, or 0.1 mg/pellet) were implanted in order to provide a continuous and constant drug delivery over 60 days (which represents 25 μg, 2.5 μg or 0.5 μg/day/100 g BW).

Indirect Calorimetry

Energy expenditure as well as the nature of substrate oxidized (carbohydrates or lipids) were investigated by indirect calorimetry. This principle is based on the determinations of CO₂, release (VCO₂) and O₂ consumption (VO₂) by each animal. It assumes that O₂ is entirely involved in substrate oxidation in the respiratory chain (leading to water production) while CO₂ release is related to substrate decarboxylation (in the Kreb's cycle). These measurements allow assessing energy expenditure (EE) and respiratory quotient (VO₂/VCO₂, RQ). EE represent the absolute energy dissipation during rest and activity. RQ is a relative measurement indicating the ratio of carbohydrate versus lipid involved in oxidative pathway. A ratio of 1.0 indicates exclusive carbohydrate oxidation while a ratio of 0.7 indicates exclusive lipid oxidation. Each value between these two extreme values indicates the relative proportion of each substrate (of note protein oxidation was not evaluated). As an example, RQ approaches 0.7 during fasting, indicating lipid oxidation, conversely after feeding RQ increases close to 1 indicating carbohydrate oxidation resulting from food intake and blood insulin rise. Likewise, animals fed high-carbohydrate diets have higher RQs than those fed high-fat diets.

The indirect calorimetry system (Panlab, Barcelona, Spain) consists of cages, pumps, flow controllers, valves, and analyzers. It is computer-controlled in order to sequentially measure O₂ and CO₂ concentrations as well as air flow in four separate cages allowing four simultaneous determinations. Rats are isolated in one of the four metabolic chambers, and room air is used as a reference to monitor ambient O₂ and CO₂ concentrations periodically.

At predefined intervals, the computer sends a signal to store differential CO₂ and O₂ concentrations, flow rate, allowing computing VCO₂, VO₂, RQ, and EE (Weir equation) with data acquisition hardware (Metabolism, Panlab, Barcelona, Spain).

Body Composition, Blood, and Tissue Sampling

At the end of the experimental period, animals were sacrificed by decapitation, in order to avoid the well-known effects of general anesthetics on mitochondrial metabolism. Blood, samples were immediately collected and plasma was frozen for subsequent determination of serum metabolites and hormones. Liver, muscles and fat depots were quickly excised and weighed. Liver median lobe was rapidly freeze-clamped. Muscles (plantaris, soleus and gastrocnemius) were frozen in isopentane precooled in liquid nitrogen. Mesenteric fat consisted, of adipose tissue surrounding the gastro-intestinal tract from the gastro-oesophageal sphincter to the end of the rectum with special care taken in distinguishing and removing the pancreas. Retroperitoneal fat pad was taken as the distinct depot behind each kidney along the lumbar muscles. Epididymal fat consisted, of adipose tissue on top of the epididymis. For subcutaneous depot measurement, a rectangular piece of skin was taken on the right side of each animal from the median line of the abdomen between the spine and the right hip to the first rib. Interscapular brown adipose tissue was removed and dissected free from adjacent muscles and white adipose tissue. The heart ventricles, the right kidney and the spleen were also excised, weighed and frozen.

Mitochondrial Isolation

The major part of the liver and the red part of each quadriceps were rinsed, and chopped into isolation medium (250 mM sucrose, 20 mM Tris-HCl and 1 mM EGTA-Tris, pH 7.4). Nuclei and cell debris were removed, by centrifugation at 800 g for 10 min. Mitochondria were then isolated from the supernatant by spinning twice at 8,000 g for 10 minutes. The mitochondrial pellet was resuspended in 0.5 ml of isolation buffer and kept on ice. Mitochondrial protein was measured by the bicinchoninic acid method (Pierce, Rockford, Ill.). The final mitochondrial suspensions were maintained on ice and were used for measurements of oxygen consumption rate and reactive oxygen species (ROS) production.

Mitochondrial Oxygen Consumption

The rate of mitochondrial oxygen consumption (JO₂) was measured at 30° C. in an incubation chamber with a Clark-type O₂ electrode filled with 2 ml of incubation medium (125 mM KCl, 10 mM Pi-Tris, 20 mM Tris-HCl, 0.1 mM EGTA, pH 7.2). All measurements were performed using mitochondria (1.0 or 0.2 mg mitochondrial protein/ml for liver and skeletal muscle) incubated either with various substrates: glutamate/malate (5 mM/2.5 mM) and succinate (5 mM), alone or in combination, palmitoyl carnitine (55 μM) and octanoyl carnitine (100 μM). For each substrate, JO₂ was recorded in the presence of the substrate alone (State 2) and following the addition of 1 mM ADP (state 3). Oligomycin (1.25 μg/mg protein) was added to the mitochondrial suspension to determine the non-phosphorylating respiratory rate (state 4). The incubation medium was constantly stirred with a built-in electromagnetic stirrer and bar flea. The efficiency of the mitochondrial oxidative phosphorylation was assessed by the state 3/state 4 ratio which measures the degree of control imposed on oxidation by phosphorylation (respiratory control ratio, RCR).

Oxidative Phosphorylation Efficiency

ATP/O ratios with 5 mM glutamate/2.5 mM malate/5 mM succinate or octanoyl-carnitine (100 μM) as respiratory substrates were determined from the ATP synthesis rate (J_{ATP}) versus respiratory rate JO₂ with an ADP regenerating system based, on hexokinase (EC 2.7.1.1) plus glucose. J_{ATP} and JO₂ were measured as described above in a medium containing 125 mM KCl, 1 mM EGTA, 5 mM Tris-Pi, 20 mM Tris-HCl, 0.1% fat free BSA (pH 7.2). J_{ATP} was determined from glucose 6-phosphate formation in presence of 20 mM glucose, 1 mM MgCl₂, and 125 μM ATP. JO₂ and J_{ATP} were modulated by addition of increasing concentrations of hexokinase (Nogueira et al, J Bioenerg Biomemb., 34:55-66, 2002).

Enzymatic Activities

Measurement of the specific activity of the respiratory-chain complex I, II and IV was performed spectrophotometrically. A total of 8-10 μg of mitochondrial proteins were required to determine the activity of complex I and II, and 4 μg were used for complex IV. Enzyme activity was expressed as nmoles of reduced or oxidized substrate per min and per mg of mitochondrial protein.

Measurement of complex 1 (rotenone-sensitive NADH-ubiquinone oxidoreductase, EC 1.6.99.3): The assay was performed using decylubiquinone (100 μM) as electron acceptor and NADH (200 μM) as a donor, in a 10 mM KH₂PO₄/K₂HPO₄ buffer (pH 7.5) containing BSA (3.75 mg/ml), and in the presence of KCN (2 mM) and antimycin A (7.5 μM). The oxidation of NADH was then measured at 340 nm before and after the addition of rotenone (4 μM), allowing the calculation of the rotenone-sensitive specific activity of complex I.

Measurement of complex II (succinate-ubiquinone reductase, EC 1.3.99.1): Succinate-ubiquinone oxidoreductase activity was quantified by measuring the decrease in UV absorbance due to the reduction of DCPIP (100 μM) at 600 nm. The measurement was performed in a medium containing 50 mM KH₂PO₄/K₂HPO₄ (pH 7.5) in the presence of decylubiquinone (100 μM), rotenone (2 μM) and KCN (2 mM).

Measurement of complex IV (cytochrome c oxidase, EC 1.9.3.1): The assay was performed by measuring cytochrome c (100 μM) oxidation at 550 nm in a 50 mM KH₂PO₄/K₂HPO₄ buffer (pH 7.0).

Citrate synthase activity was determined by measuring the UV absorbance at 412 nm due to the formation of the ion mercaptide in the presence of oxaloacetate dinitrothiobenzoïque acid and acetyl-CoA in a 150 mM Tris buffer pH 8 (Garait et al, Free Rad Biol Med, 2005).

Mitochondrial glycerol 3-phosphate dehydrogenase (mGPdH) activity was measured on the supernatant of isolated mitochondria after three cycles of freezing-thawing. Forty μg of mitochondria were incubated in a KH₂PO₄/K₂HPO₄ buffer (50 mM, pH 7.5) containing 9.3 μM of antimycin A, 5 μM of rotenone and decylubiquinone (50 μM). The reduction of 50 μM dichloro-indophénol (DCIP) by mGPDH was measured spectrophotometrically at 600 nm at 37° C. and enzymatic activity was expressed as μmol.min⁻¹.mg prot⁻¹.

Cytochromes

Cytochromes content of the mitochondrial respiratory chain was measured in parallel experiments by comparing the spectra of fully oxidized, (potassium ferricyanide) versus fully reduced (few crystals of sodium dithionite) cytochromes. Knowing the contributions in absorbance of each cytochrome to the major maxima and minima of each of the other cytochromes, a set of 4 simultaneous equations with 4 unknowns can be derived and concentration of each cytochrome can be calculated (Williams, Arch Biochem Biophys.; 107: 537-43, 1964)

Hepatocytes Isolation

Wistar rats fasted for 20-24 h were anesthetized with sodium pentobarbital (10 mg/100 g body wt i.p.), and the hepatocytes were isolated according to the method of Berry and Friend (J. Cell. Biol. 43: 506-520, 1969) as modified by Groen et al. (Eur. J. Biochem. 122: 87-93, 1982). Briefly, the portal vein was cannulated, and a 2-min anterograde perfusion with Ca²⁺-free Krebs-Ringer bicarbonate buffer (25 ml/min; 37° C., pH= 7.4, continuously gassed with 95% O₂-5% CO₂) was performed, to remove blood, from the liver, A 10-mm retrograde perfusion (25 ml/min) through the posterior vena cava was started with the same perfusion medium. Subsequently, a recirculating perfusion was performed (20 min at 40 ml/min) with 100 ml Krebs-Ringer medium supplemented with 0.25 mg/ml collagenase (type IV, Sigma, St. Louis, Mo.). The liver was then cut and shaken in the perfusion medium for 2 min under constant gassing (95% O₂-5% CO₂). Finally, the cell suspension was filtered through nylon gauze (pore size, 120 μm), washed twice with Krebs-Ringer bicarbonate buffer containing 1.6 mM Ca²⁺, and then washed for a third time with the same buffer supplemented with 1% BSA.

Perifusion of Hepatocytes

Liver cells were perifused according to the method of van der Meer and Tager (FEBS Lett. 67: 36-40, 1976) modified by Groen et al. (Eur. J. Biochem. 122: 87-93, 1982). Hepatocytes (225-250 mg dry mass) were placed in 15-ml perifusion chambers at 37° C. and were perifused (5 ml/min) with a continuously gassed (95% O₂-5% CO₂) Krebs-Ringer bicarbonate solution (pH= 7.4) containing 0.2% BSA. The experiments were carried out in duplicate in two perifusion chambers placed in parallel. At the chamber outlet, perifusate O₂ content was monitored with Clark electrodes (Yellow Springs Instruments, Yellow Springs, Ohio) to assess O₂ uptake of the hepatocyte suspension. After 40 min, when O₂ uptake had reached a steady state, hepatocytes were perifused with increasing amount of glycerol (0.15, 0.30, 0.60, 1.2, 2.4, 4.8, and 9.6 mM), in the presence or not of 0.4 mM octanoate. At the end of each steady state of 20 min, perifusate and cells samples were collected at 2-min intervals for subsequent determination of glucose, lactate, pyruvate, acetoacetate, and β-hydroxybutyrate concentrations. Samples were stored at 4° C. and analyzed within 12 h after the end of the experiment. In addition, 300 μl of the cell suspension were sampled from the chamber for intra- and extracellular fractionation. For this purpose, mitochondrial and cytosolic spaces were separated with the digitonin fractionation method described by Zuurendonk and Tager (Biochim. Biophys. Acta 333: 393-399, 1974). Briefly, the cell suspension was placed in a 2.2-ml Eppendorf tube in an isotonic medium containing 2 mM of digitonin (Merck, Lyon, France) at 4° C. After 15 s, the tube was centrifuged for 15 s at 10,000 g to precipitate mitochondria through the underlying 800-μl layer of silicon oil (Rhodorsil 640 V 100, Rhône-Poulenc) into 250 μl HClO₄ (10% mass/vol)±25 mM EDTA. The supernatant (700 μl) was immediately removed, deproteinized with HClO₄ (5% mass/vol), and neutralized. The intracellular content was then neutralized, and kept at −20° C. for determination of intracellular metabolites (DHAP and G3P, spectrophotometry) and adenine nucleotides content (HPLC).

Western Blot Analysis

For mGPdH quantification, polyacrylamide gel electrophoresis and immunoblotting were performed as previously described (23). Briefly, lysed hepatocytes were mixed with 200 μl of buffer containing 40 mM Tris(hydroxymethyl)aminomethane pH 6.8, 1% SDS, 6% glycerol, and 1% b-mercaptoethanol. This mixture was then heated at 100° C. for 10 min, and subjected to one-dimensional sodium dodecyl sulfate (SDS)-PAGE with a 5% stacking and 12.5% resolving gels for 12 hours. After electrophoretic separation, proteins were transferred at a constant voltage to PVDF membranes. After protein transfer, the membranes were blocked for 2 h, then incubated 2 h with a monoclonal antibody specific for mGPDH (generous gift from Dr. J. Weitzel) and then exposed, to the secondary antibody (goat anti-mouse immunoglobulin G conjugated to horseradish peroxidase, Bio-Rad at a 1:10000 dilution). mGPDH were visualized by the enhanced chemiluminescence detection method (RPN 2106, Amersham), Scanning with a densitometer performed, quantification of bands from blots and the data were expressed numerically as integrated optical density arbitrary units.

RNA Purification and Reverse Transcription-Coupled PCR

Total RNA were extracted from tissue using Tripure RNA Isolation reagent (Roche Diagnostics). Concentration and purity were verified by measuring optimal density at 260 and 280 nm. Their integrity was checked by 1% agarose gel electrophoresis (Eurobio). mRNA concentrations were measured by semi-quantitative reverse transcription polymerase chain reaction (RT-PCR) using β actin as reference. Primer sequences are shown in table 1. For each target mRNA, a RT was performed from 0.1 μg of total RNA with 100 U of M-MLV Reverse Transcriptase (Promega), 5 μL of M-MLV RT 5× buffer, 20 U of RNasin Ribonuclease Inhibitor, 12 pmoles of deoxynucleoside triphosphate and 15 picomoles of the specific antisense primer, in a final volume of 25 μL. The reaction consisted in 5 min at 70° C. (RNA and antisense primer), then 60 min at 42° C. (all mix) followed by 15 min at 70° C. After chilling, 5 μL were used for PCR reaction. The 5 μL of RT medium were added to 45 μL of PCR mix (5 μL 10× REDTaq PCR buffer) containing 6 picomoles of MgCl2, 8 picomoles of deoxynucleoside triphosphate, 2.5 U of REDTad DNA polymerase (Sigma), 15 picomoles of corresponding antisense primers and 22.5 picomoles of sense primers. The PCR conditions were: 2 min at 94° C. followed by 28 cycles, 35 cycles or 18 cycles for UCP3, UCP2 and β actin respectively (1 cycle=1 min at 94° C., 1 min at 60° C., 1 min at 72° C.). PCR was ended by 10 min at 72° C. Products were analysed on 2% agarose gel prestained with ethidium bromide. For quantitation of relative bands intensities, pictures were taken with a Camera DC120 (Kodak) and the ratio of each target to β actine was determined for each sample with Kodak Digital Science 1D 2.0 (Kodak Scientific Imaging System).

### 2. Results

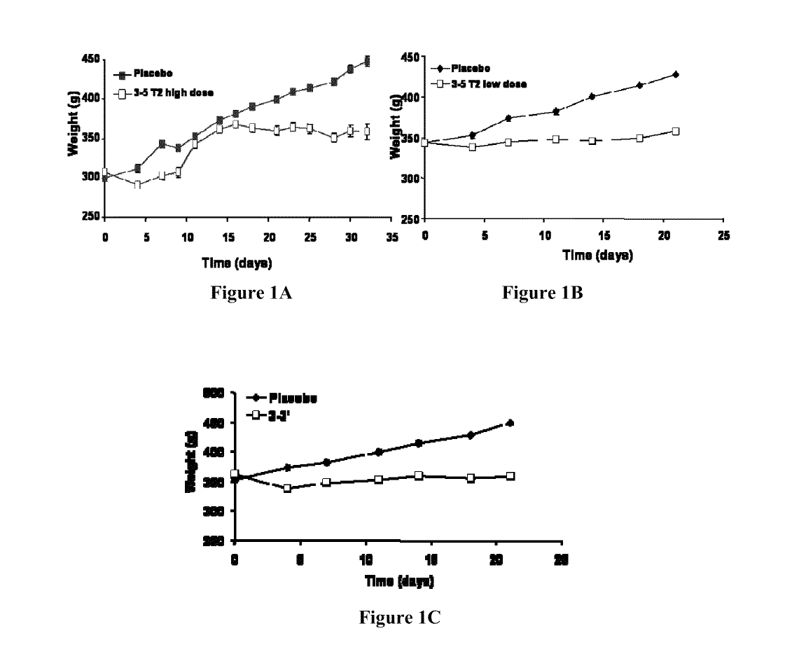

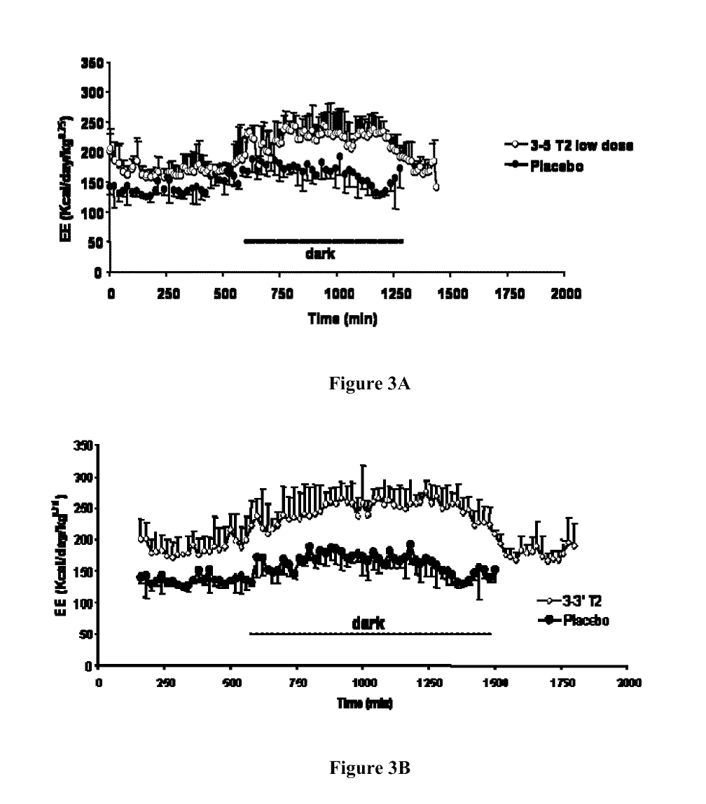

As shown in FIGS. 1 (A,B and C), control (placebo treated) Wistar rat body exhibit a normal growth rate of 150 g over 34 (1A) and about 60 g over 21 days (1B). Treated animals with high dose of either 3,5-T2 (FIG. 1A) or 3,3′-T2 (FIG. 1C) did not show similar weight gain. In the group treated with 3,5-T2, a biphasic curve was observed with a weight gain between the 10^{th} and the 15^{th} day, while after the body mass did not change as it was the case with 3,3′-T2 treatment either at low 3,5-T2 (FIG. 1B) or high 3,3-T2 dosage (1C).

This indicates a very powerful prevention of normal weight gain in these young adult animals.

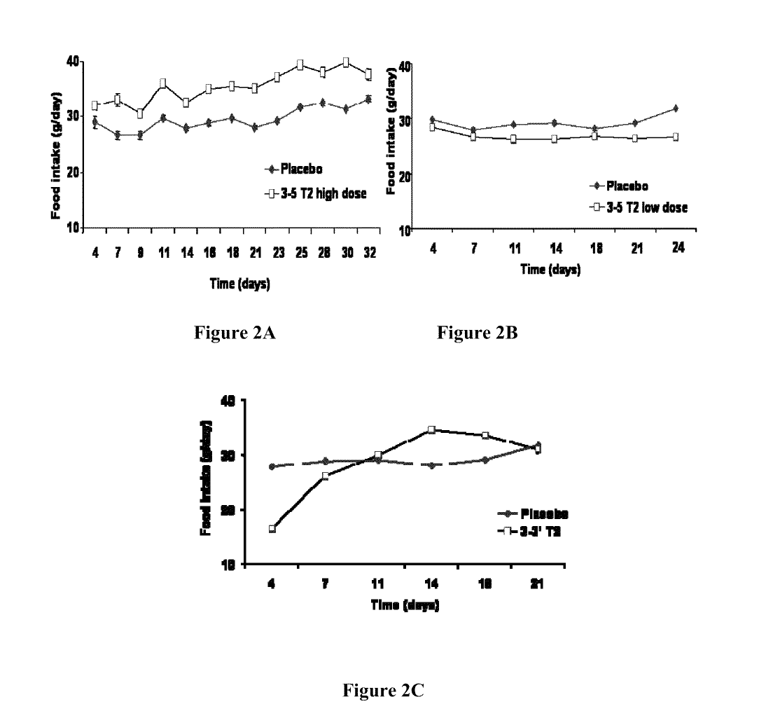

As shown in FIGS. 2 (A, B and C), the food intake of placebo group was stable over the experimental period around 30 g of food, per day. While food intake was almost not affect at low dose 3,5-T2 treatment (or slightly decreased, FIG. 2B) a clear increase was observed in high dosage 3,5-T2 group (FIG. 2A) and a biphasic effect was observed with 3,3′-T2: the initial decrease was followed by an increase after the 10^{th} day.

Hence the decrease in body weight in both groups of treated animals was associated with either no change or an increase in food intake.

The energy expenditure (EE) of rats was assessed by indirect calorimetry (see material and method section) and values were analyzed over a period of 24 hours (=1440 minutes). Two groups of animals, treated with placebo (FIGS. 3A and 3B), low dose 3,5-T2 (FIG. 3A) or 3,3′-T2 (FIG. 3B), exhibited days/nights variations due the classical nocturnal activity and eating behavior of these rodents contrasting with the quiet diurnal period. Both groups of treated, rats exhibited a dramatic increase in energy expenditure reaching 25 to 30 of the control values. The difference appeared more marked in the group of 3,3′-T2, which received a higher amount of hormone (25 μg/100 g bw) as compared to 3,5-T2, which received only 2.51 μg/100 g bw).

This very important result indicates that the metabolic expenses are largely increased by the two treatments both during the nocturnal and the diurnal periods.

The respiratory quotient (RQ) is defined as the ratio between released, carbon dioxide to consumed oxygen: VCO₂/VO₂. It is largely accepted that this ratio indicate the origin of oxidized substrates (carbohydrate versus lipids). This value is equal to 1 if carbohydrates represent the exclusive source of energy and 0.7 were lipids represent the unique energetic substrate.

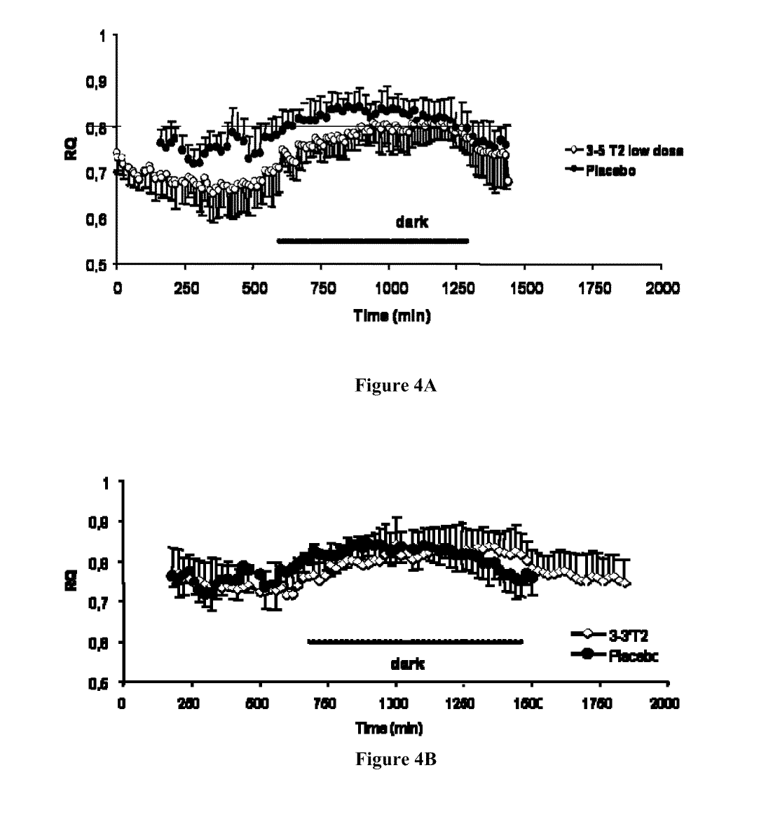

As already shown for the EE, RQ also varies between day and night (FIGS. 4A and 4B). It is higher during the night, when animals are eating and therefore oxidizing more carbohydrates. Conversely during the diurnal period. RQ is lower indicating a fasting sate were lipids are the predominant substrates. Regarding 3,5-T2 low dose (FIG. 4A), it appears that RQ is lower than RQ with placebo treatment during the day and the first part of the night and almost identical at the en of the night. In general, and taken into account the day/night variations, RQ is lower in the group with low 3,5-T2 as compared to high 3,3′-T2. This would indicate either a higher proportion of carbohydrate or, more likely, a net lipid synthesis from carbohydrate (leading to a RQ value higher than 1) the value presented by these animals being the sum of substrate oxidation and substrate (lipid synthesis) during the fed state.

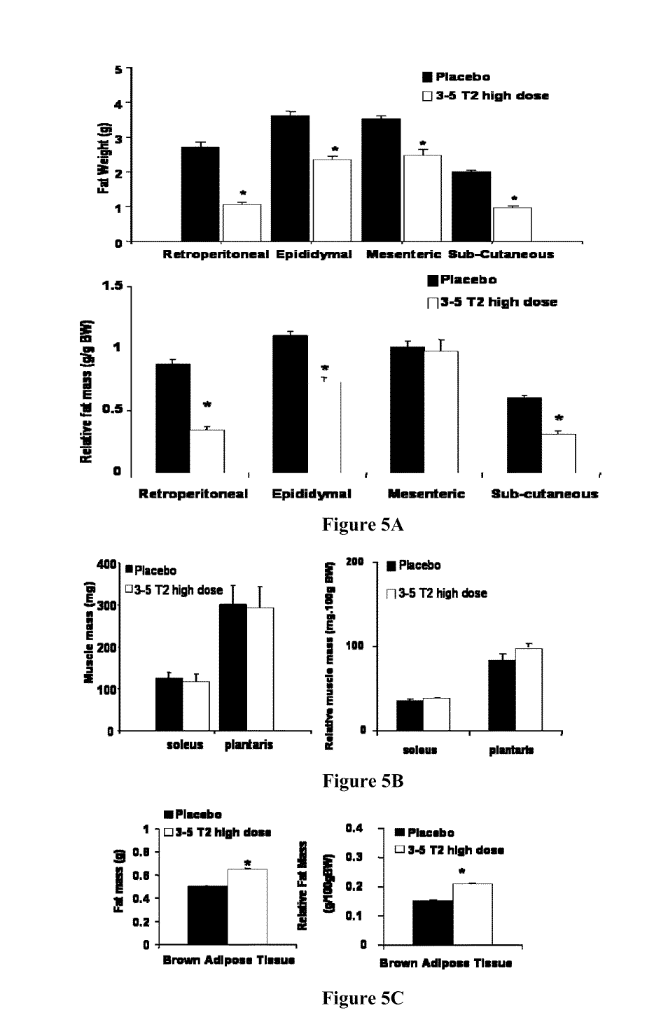

The change in body composition of rats treated with high dose 3,5-T2 (FIG. 5A) or placebo are presented both as absolute values (g) or as percentage of total body mass since the two groups of animals did not exhibit the same mass after three weeks (see FIG. 1A).

All localizations of fat masses were significantly lower (p<0.01) in 3,5-T2 group as compared to placebo (FIG. 5A). This difference was very substantial whatever the data are expressed as absolute or relative values (excepted relative mesenteric mass). Interestingly muscle mass was not affected at all (FIG. 5B), while brown adipose tissue, a tissue known to be involved in metabolic efficiency and heat production, was significantly increased in 3,5-T2 treated animals (FIG. 5C).

These results clearly indicate that the decrease in body mass after 3,5-T2 treatment is purely due to a loss of fat mass, the lean body mass being not affected.

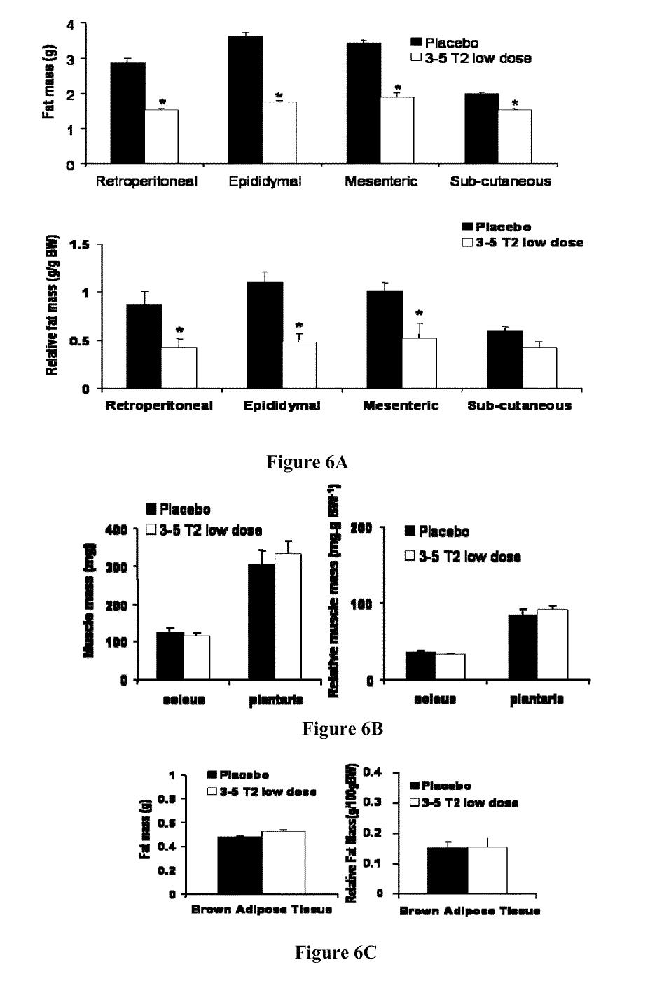

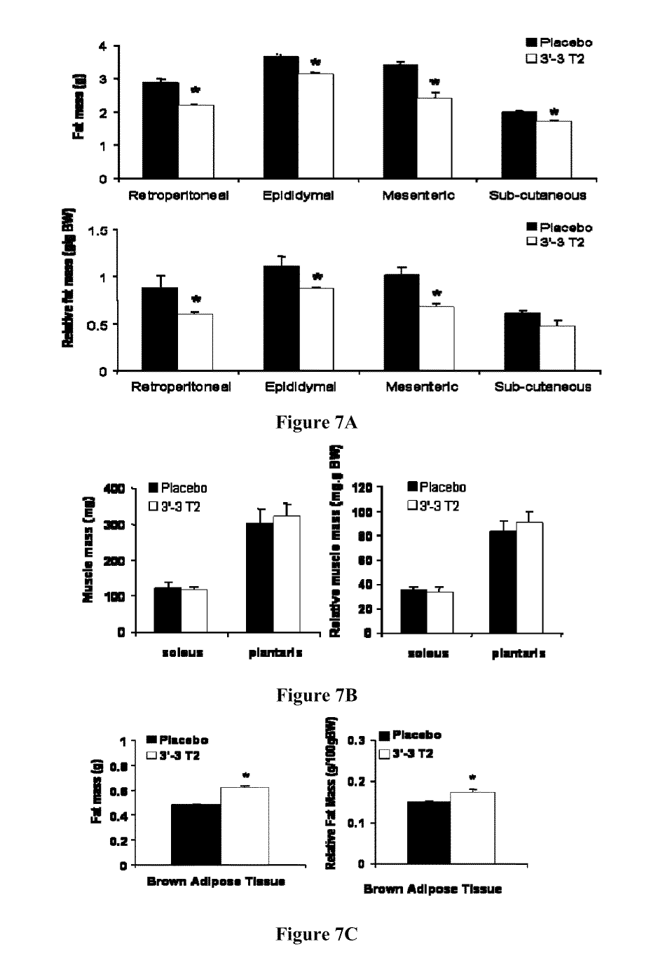

Similar results are obtained with 3,5-T2 low dose (FIGS. 6A, 6B and 6C) or with 3,3′-T2 (FIGS. 7A, 7B and 7C) leading to reach the same conclusion regarding their effect on fat mass (significantly decreased) and lean body mass (not affected). Interestingly brown adipose tissue was significantly increased by the 3,3′-T2 treatment (high dose), but not by 3,5-T2 at low dose.

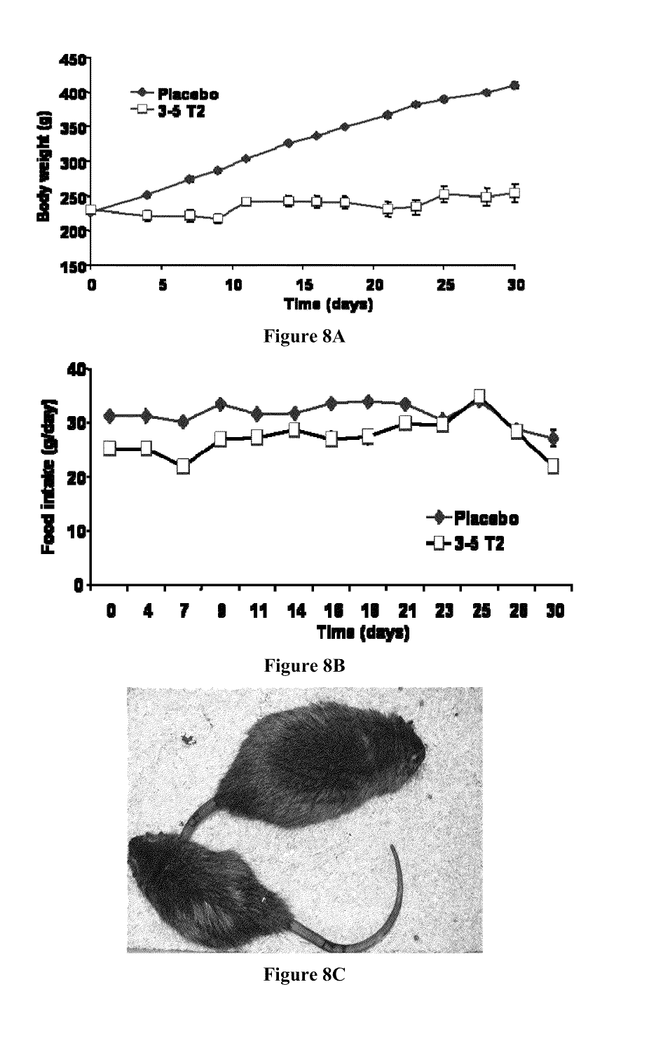

Very interestingly genetically obese rats (i.e. with a genetic defect in the leptin signaling pathway Fa/Fa) a strong prevention of animal growth was also observed (+170 g over 30 in placebo versus no significant growth in the low 3,5-T2 group (FIG. 8A) while the food intake was significantly lower as compared to placebo (FIG. 8B), The difference in both animal features is impressive (FIG. 8C).

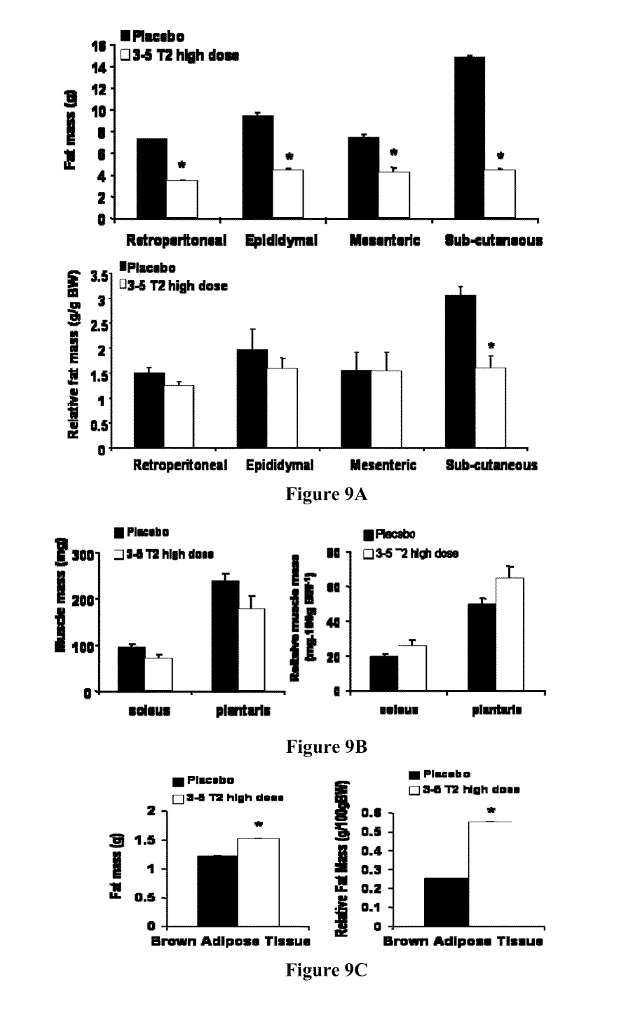

Similarly to what was observed in Wistar rats, absolute values of fat deposit, in all localizations, were significantly lower in 3,5-T2 treated animals, the decrease being particularly dramatic regarding the subcutaneous localization (FIG. 9A). Muscle mass was not affected (FIG. 9B) and brown adipose tissue was significantly increased (FIG. 9C).

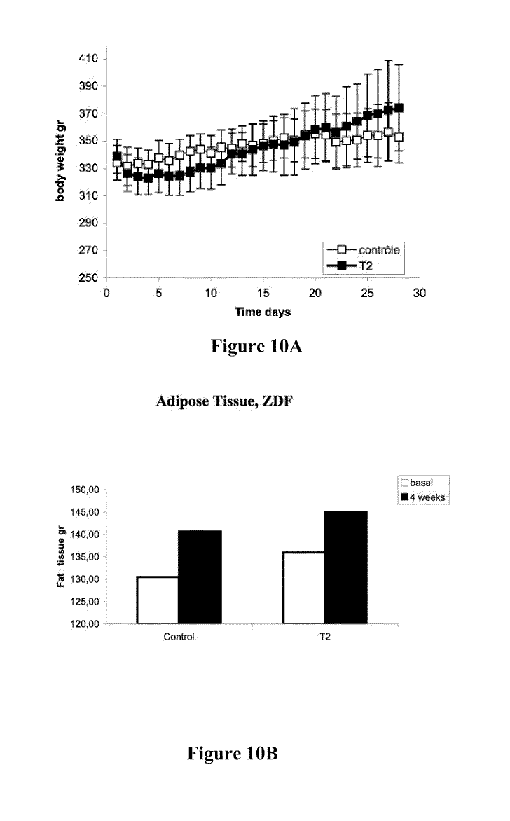

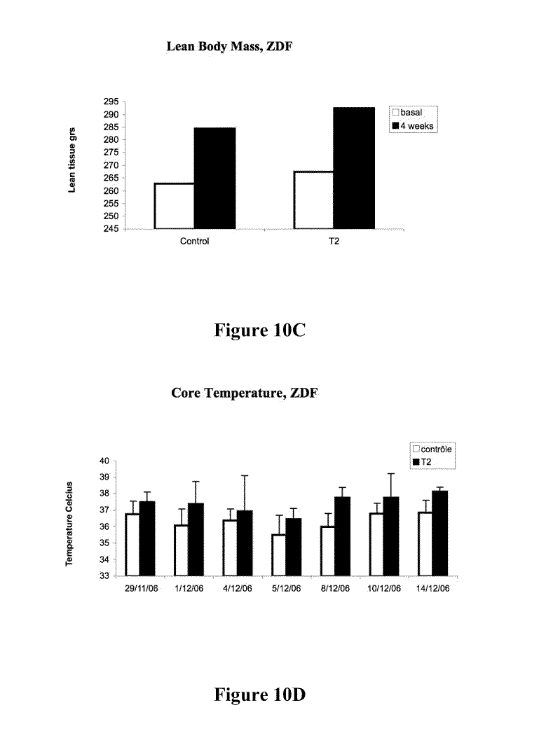

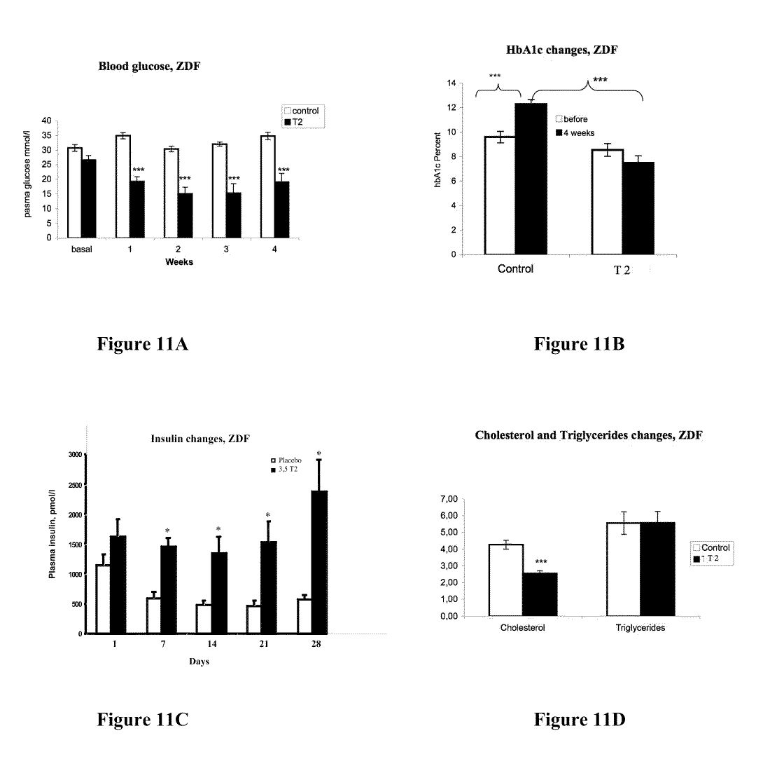

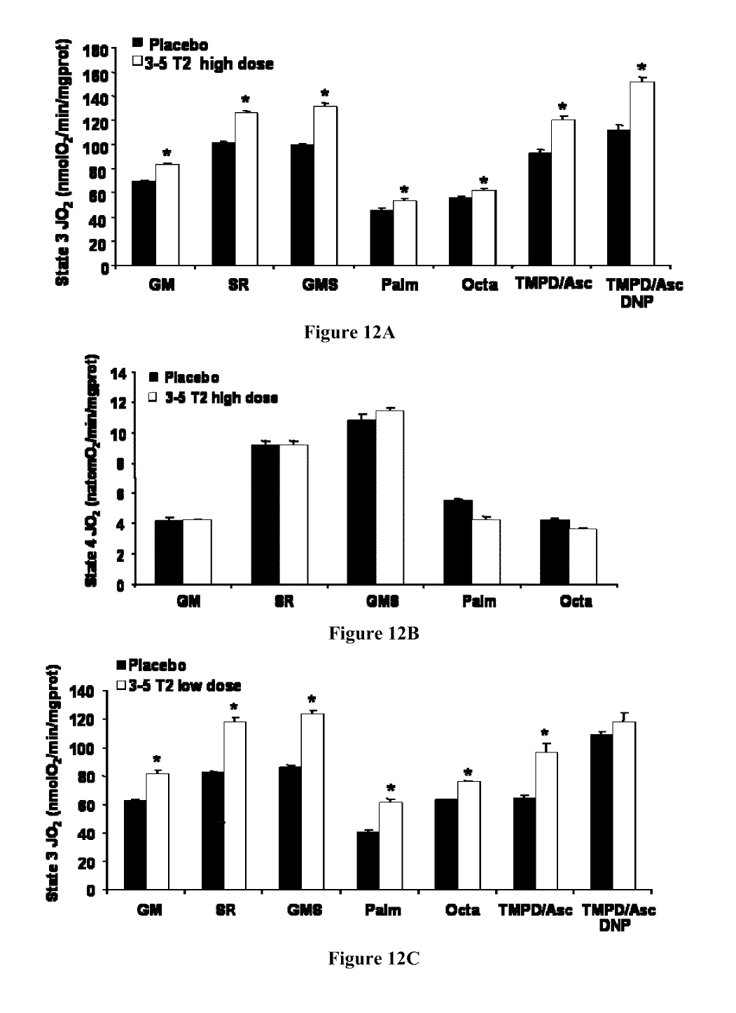

By contrast, when ZDF rats were investigated, it was failed to find, a significant effect of 3,5-T2 high dose on animal growth (FIG. 10A), while fat mass gain was less (FIG. 10B) and lean body mass increase was higher (FIG. 10C) as compared to placebo. Furthermore the core temperature of the treated animals was higher as compared to placebo (FIG. 10D). It is important to note that in this model of diabetic animals the insulin secretion is progressively impaired (see below), probably because a progressive “gluco-toxicity” of high plasma glucose towards pancreatic beta cells. Hence the growth rate of these diabetic animals is much less than that of their obese non-diabetic littermates (compared the growth rate of placebo group in FIGS. 8A (+170 g) and 10A (almost no change)). Interestingly, there is a modest but clear growth in the 3,5-T2 group suggesting a lesser toxic effect.

As shown in the FIG. 11D, at the end of the treatment, cholesterol was significantly lower in the group of ZDF rats treated with 3,5-T2, while triglycerides levels were not different between the two groups.

The effect of both treatments (3,5-T2, high and low doses, or 3,3′-T2) on the efficacy of the coupling between oxidation and phosphorylation at the level of liver mitochondrial respiratory chain were evaluated (FIG. 12). The different conditions glutamate/malate, succinate-rotenone, glutamate/malate/succinate, palmitoylCoA, octanoylCoA indicate the different substrates provided to the respiratory chain. FIGS. 12A (3,5-T2 high dosage), 12C (3,5-T2 low dosage) and 12E (3,3′-T2) represent the maximal respiratory rate of liver mitochondria achieved in phosphorylating condition (i.e. in the presence of ADP) with the various substrate supply: TMPD ascorbate investigate complex 4 (cytochrome c oxidase) without or with uncoupling state by DNP. Schematically in all conditions treatments were responsible for a very significant increased respiratory rate indicating that the treatments increased the maximal respiratory capacity for all substrates, including fatty acids.

Respiratory rates of non-phosphorylating mitochondria (i.e. in the presence of oligomycin) of the different groups (3,5-T2 high and low doses or 3,3′-T2: FIGS. 12B, 12D and 12F respectively) of treated animals versus placebo were measured. As compared to placebo, respiration was substantially higher in the low 3,5-T2 group only.

Very interestingly completely different results were obtained with muscle mitochondria. Indeed 3,5-T2 low and high dosage and 3,3′-T2 failed to substantially affect both phosphorylating (state 3, FIGS. 14A, 14B and 14C) and non-phosphorylating (state 4, FIGS. 15A, 15B and 15C) states.

Hence this indicated that although both 3,5-T2 at low and high dosage and 3,3′-T2 exhibit a powerful effect on liver mitochondria, almost no effect was found on muscle mitochondria despite the feet that the drug was administered to every tissue (subcutaneous progressive release from the pellet).

FIG. 15 show the effect of 3,5-T2 and 3,3′-T2 at the end of the treatments on glucose in Wistar (FIG. 15A) and Zucker (FIG. 15 B) rats. In these non-diabetic animals, treatments were only responsible for minor changes, either increase in Wistar or decrease in Zucker.

Triglycerides (FIGS. 16A and 16B), and cholesterol (FIGS. 17 A and 17B) were decreased with all treatments in Wistar, Zucker and ZDF rats, while free fatty acids (FIGS. 18A and 18B) were increased, indicating a high rate of lipolysis and fatty acid oxidation as it was already suggested by the data obtained with indirect calorimetry. HDL (FIGS. 19A and 19B) were decreased only in Zucker or ZDF rats. Plasma fatty acids were higher as it is observed in animals.

Finally data presented in FIG. 20 (A and B) directly investigating the efficiency of oxidative phosphorylation (ATP/oxygen ratio) show the 3,5-T2 lowers the yield of ATP synthesis with fatty acids (both palmitoyl- and octanoyl-CoA) and succinate (FIGS. 20 B, 20C and 20D).

In conclusion, the dramatic effect observed in the body mass is completely explained by the decreased fat mass, while the lean body mass (muscle mass) seems not affected. This effect, which is observed despite increased food consumption, is due to an increased energy-expenditure, which was substantiated by indirect calorimetry measurement. Since the normal diet of these animals is rather poor in lipid content (4-5%) the increase fat oxidation is achieved, at the expense of the fat storage as shown by the strong decrease in fat mass and also probably by a de novo lipogenesis, an expensive pathway, which might explain the higher RQ observed in the fed period. The data concerning the overall increase in energy metabolism (indirect calorimetry) are in very good agreement with the data obtained in liver isolated mitochondria indicating the probably occurrence of energy wasting process at the level of the respiratory chain and ATP synthesis associated with a significant higher maximal respiratory capacity. Most interestingly none of these effects was observed in muscle mitochondria indicating that the wasting process affects more the liver than the muscle mass and concerns lipid oxidation.

Hence in total both 3,5-T2 at both high and low dosages and 3,3′-T2 enhance lipid oxidation and energy expenditure leading to a marked decrease in the mass of adipose tissue only.

### Example 2

### Use of the 3,5-T2 Hormone for the Treatment of Diabetes

### 1. Material and Methods

The Material and Methods are those described, in Example 1.

Animals

Rats were genetically obese normoglycemic (Zucker or Fa/Fa), 10-11 week-old diabetic rats (ZDF) or genetic non-overweight diabetic (type 2 diabetes) rats (Goto-Kakizaki (GK) model).

Blood Sampling

The day of the study, after a fasted period overnight (18 h), blood samplings will be taken in awake rats from the tail vein.

Blood Parameters

The following biochemical parameters were analyzed: glycemia, insulinemia, HbA1c, TG and Cholesterol.

Thyroid Stimulating Hormone (TSH) and Thyroxine (T4) were measured by radioimmunoassay with rat standards (RPA 554 Amersham bioscience, RIA FT4-immunotech, for TSH and T4 respectively).

Insulin levels were determined with commercial kits (Linco Research).

Glucose and 3-hydroxybutyrate (3-HB) were measured, enzymatically and non esterified fatty acid (NEFA) by colorimetric assay (Wako Chemicals).

Triglycerides and cholesterol were measured by classical routine automate apparatus.

### 2. Results

As shown in FIGS. 11A and 15B, a high dosage of 3,5-T2 results in dramatic decrease in plasmatic glucose concentration of ZDF rats, already after one week, the effect being present over the 4 weeks of the experimental period. This lowering blood glucose effect is confirmed by the significant decrease in the glycated hemoglobin (HbA1c) a good marker of chronic hyperglycemia (FIG. 11S). This effect is accompanied by the maintenance of insulin concentration at a high level in the treated group contrasting with the progressive decrease in time of insulin levels in the placebo group (FIG. 11C). The decrease in insulin level in control ZDF rats is explained by a decrease in insulin secretion related to a toxic effect of high glucose on pancreatic beta-cells. Hence the maintenance of a high insulin level throughout the whole experimental period indicates a protection of beta-cells, which could be due to either the lowering of blood glucose, a prevention of cell death or a regeneration process. Anyway, this result indicates a higher insulin secretion by pancreas in the treated group.

Furthermore, GK rats treated with low dosage 3,5-T2 (2.5 μg/100 g BW) during 10 days exhibit a significant decrease in blood glucose concentration of −24±6% (n=5, p<0.01) as compared to control animals (blood glucose concentration of 2.64±0.21 g/l (n=22)). This decrease in blood glucose already after 10 days of treatment indicates an improvement of the hyperglycemic (diabetic) status in this model where high glycemia is believed to be due to both insulin deficiency and insulin resistance.

In conclusion, 3,5-T2 is responsible for a dramatic decrease in blood glucose, a feature accompanied by an increase in insulin in a model of severe “type-2” diabetes (ZDF rat) indicating an increase in insulin sensitivity as well as insulin secretion. This important feature is novel since so far treatments were found to only delay the onset of diabetes in such animals but not to correct it when installed.

1-14. (canceled)

15. A method for the treatment of pathologies chosen among hyperglycemia, insulin resistance, beta pancreatic cell insufficiency or related pathologies comprising the administration in a patient in a need thereof of a pharmaceutically effective amount of at least one hormone chosen among
3,5-diiodothyronine,
3′,3-diiodothyronine,
3′,5-diiodothyronine,
3′-iodothyronine,
3-iodothyronine and
5-iodothyronine.

16. The method according to claim 15, wherein said hormone is chosen among 3,5-diiodothyronine, 3′,3-diiodothyronine and 3′,5-diiodothyronine.

17. The method according to claim 15, for the treatment of diabetes, particularly type 1 or 2 diabetes.

18. A pharmaceutical composition comprising as active substance at least one hormone chosen among 3,5-diiodothyronine, 3′,3-diiodothyronine, 3′,5-diiodothyronine, 3′-iodothyronine, 3-iodothyronine or 5-iodothyronine, in association with a pharmaceutically acceptable vehicle suitable for an administration via a subcutaneous or transcutaneous route.

19. The pharmaceutical composition according to claim 18, wherein said pharmaceutically acceptable vehicle allows a continuous, preferably constant, release, of said active substance.

20. The pharmaceutical composition according to claim 18, in a suitable form for the release of about 0.01 μg/kg/day to about 250 μg/kg/day, particularly about 0.01 μg/kg/day to about 25 μg/kg/day, particularly about 0.1 μg/kg/day to about 15 μg/kg/day of active substance, more particularly about 0.1 μg/kg/day to about 5 μg/kg/day of active substance, most particularly about 0.1 μg/kg/day to 1 μg/kg/day of active substance.

21. The pharmaceutical composition according to claim 18, wherein said pharmaceutically acceptable vehicle allows a continuous, preferably constant, release, of said active substance and said pharmaceutical composition is in a suitable form for the release of about 0.01 μg/kg/day to about 250 μg/kg/day, particularly about 0.01 μg/kg/day to about 25 μg/kg/day, particularly about 0.1 μg/kg/day to about 15 μg/kg/day of active substance, more particularly about 0.1 μg/kg/day to about 5 μg/kg/day of active substance, most particularly about 0.1 μg/kg/day to 1 μg/kg/day of active substance.

22. The pharmaceutical composition according claim 18, comprising by dosage unit about 5 μg to about 1.5 g of active substance, particularly about 75 mg to about 750 mg of active substance.

23. The pharmaceutical composition according claim 18, comprising by dosage unit about 5 μg to about 1.5 g of active substance, particularly about 75 mg to about 750 mg of active substance and wherein said pharmaceutically acceptable vehicle allows a continuous, preferably constant, release, of said active substance.

24. The pharmaceutical composition according claim 18, wherein said pharmaceutically acceptable vehicle is a chemical, such as alcohol, used to enhance skin penetration.

25. A method for the treatment of pathologies chosen among:
hyperglycemia, insulin resistance, beta pancreatic cell insufficiency, diabetes, or related pathologies,
obesity, overweight or related pathologies, hypercholesterolemia, hypertriglyceridemia, dyslipidemia, alcoholic and non alcoholic hepatic steatosis, atherosclerosis, hepatopathies associated to a dysmetabolism, cholecystopathies, deposit of subcutaneous fat, particularly cellulite or vasomotor rhinitis
comprising the administration in a patient in a need thereof of a pharmaceutically effective amount of at least one hormone chose among 3,5-diiodothyronine, 3′,3-diiodothyronine, 3′,5-diiodothyronine, 3′-iodothyronine, 5′-iodothyronine, 3-iodothyronine or 5-iodothyronine,
said hormone and said pharmaceutically acceptable vehicle being under a suitable form for an administration via a subcutaneous or transcutaneous route.

26. The method according to claim 25, for the treatment of hyperglycemia, insulin resistance, beta pancreatic cell insufficiency or related pathologies.

27. The method according to claim 25, for the treatment of diabetes, particularly type 1 or 2 diabetes.

28. The method according to claim 25, wherein said pharmaceutically acceptable vehicle allows a continuous, preferably constant release of said active substance.

29. The method according to claim 25, wherein said hormone and said pharmaceutically acceptable vehicle are in a suitable form for in a suitable form for the release of about 0.01 μg/kg/day to about 250 μg/kg/day, particularly about 0.01 μg/kg/day to about 25 μg/kg/day, particularly about 0.1 μg/kg/day to about 15 μg/kg/day of active substance, more particularly about 0.1 μg/kg/day to about 5 μg/kg/day of active substance, most particularly about 0.1 μg/kg/day to 1 μg/kg/day of active substance.

30. Product comprising:
at least one hormone chosen among 3,5-diiodothyronine, 3′, 3-diiodothyronine, 3′, 5-diiodothyronine, 3′-iodothyronine, 3-iodothyronine or 5-iodothyronine, and
at least one active substance activating the pancreatic secretion of insulin, particularly chosen among antidiabetic oral drugs, or susceptible of slowing the digestive absorption of glucose,
as a combination product for a simultaneous, separated or sequential use intended for the treatment of diabetes.
